(19) United States
(12) Patent Application Publication
(10) Pub. No.: US 2026/0306736 A1
(43) Pub. Date: Oct. 8, 2026

(54) A medicament tube arrangement

(71) Applicant: SHL Medical AG, Zug (CH)

(72) Inventors: Martin Michael Coyne, III, Boonton, NJ (US); Christopher James Franzese, Boonton, NJ (US); Thomas Daniel James, Baltimore, MD (US); Katherine Ford Brigham, Boonton, NJ (US); Madeleine Hayden Anderson, Boonton, NJ (US); Matthew Conner D'Auria, Boonton, NJ (US)

(21) Appl. No.: 19/473,834

(22) PCT Filed: Apr. 5, 2024
(86) PCT No.: PCT/EP2024/059308

§ 371 Date: Oct. 9, 2025

Related U.S. Application Data
(60) Provisional application No. 63/460,363, filed on Apr. 19, 2023.
(60) Provisional application No. 63/523,150, filed on Jun. 26, 2023.

(30) Foreign Application Priority Data
May 19, 2023 (EP) 23174218.0

(51) Int. Cl. A61M 5/14; A61M 5/142

(52) U.S. Cl. CPC A61M 5/1418; A61M 5/1408; A61M 5/14244; A61M 2209/088

(57) ABSTRACT

The present disclosure provides a medicament tube arrangement for an infusion set. The medicament tube arrangement includes a first flexible tube structure and a second flexible tube structure for delivering medicament from a medicament reservoir. The medicament tube arrangement further includes at least one connector being configured to connect the first flexible tube structure to the second flexible tube structure. The connector is arrangeable in a first configuration in which the first flexible tube structure is arranged adjacent the second flexible tube structure and is arrangeable in a second configuration in which the first flexible tube structure is arranged spaced apart from the second flexible tube structure.

### TECHNICAL FIELD

The present disclosure generally relates to medical devices for medicament administration.

### BACKGROUND

Infusion and injection are commonplace medical procedures used to deliver a wide variety of therapeutic medicines of interest for a variety of diseases. “Infusion,” “injection,” and “administration” may be used interchangeably, taking place by subcutaneous (SC), intramuscular (IM), intravenous (IV), enteral, or other routes, also terms used interchangeably. Administration route is based on a specific medication's pharmacokinetic (PK) profile, formulation components, approved regulatory labelling, individual clinical judgment, or clinical necessity.

An infusion set including a medicament delivery device, e.g. a near-body or off-body device, may require varying lengths of tubing to deliver medication from a medicament reservoir to the desired injection or infusion site. A given patient may also receive medication(s) at multiple sites (e.g., abdomen and thigh) located at varying distances from the medicament reservoir. Comparing multiple patients, varying lengths of tubing may be required to accommodate differing anthropometrics, e.g., pediatric populations, obese populations, etcetera.

One option is to provide a single length of tubing that is longer than will be needed in most scenarios, as seen with IV tubing used in clinic. However, given a near-body medicament delivery device and more active wearer, this excess length can cause issues not seen in-clinic. The length of the tubing may be difficult to route around clothing or it may get tangled, especially in the case of multi-drug delivery with multiple tubing lumens. Exposed excess tubing may catch on objects in the external environment and unanticipated strain can lead to needle pullout. Additionally, tubing length is correlated with retained medication volume (i.e., deadspace) such that increased tubing length increases the deadspace and may require increasing filled volume above the nominal volume to deliver the nominal dose volume to the patient. This increased overfill is especially undesirable in cases where the filled medication is costly such as monoclonal antibodies (mAbs).

Another option is to manufacture multiple lengths of tubing (e.g., short, medium, long) or extension sets, a sort of “patch” cable for fluidic delivery. While this minimizes the aforementioned concerns, it increases the requirements on manufacturing and dispensing workflows to ensure each patient receives a suitable length. It also requires additional manufacturing capability and inventory to stock different items, making supply chain more complex than needed. Most importantly, each length of tubing will retain a unique volume of medication. In the case of a 100 mL infusion as an example, patients with a “short” tube set will receive a larger dose than a patient using a “long” tube set owing to the retained medication, varying dose delivery and dose accuracy between patients and tubing sets. This is clinically undesirable, as it may be a latent under- or over-delivery of medication.

Moreover, tubing management is a significant challenge, as each tubing must be clearly traced from one end to the other to assure medication flows to the desired injection or infusion site. Tubing also becomes easily tangled, especially when multiple tubing are used.

Therefore, a tubing management solution is needed that will suit a wide range of patient anthropometrics and that can be reliably dispensed to the patient. Moreover, tubing management solution should be made in a way that delivers a predictable dose to each patient and does not compromise manufacturing workflows.

### SUMMARY

In view of the above, there is a need for improved medicament tube arrangement that at least partly solves the problem of the prior art.

There is hence provided a medicament tube arrangement for an infusion set comprising: a first flexible tube structure and a second flexible tube structure for delivering medicament from a medicament reservoir; at least one connector being configured to connect the first flexible tube structure to the second flexible tube structure; wherein the connector is arrangeable in a first configuration in which the first flexible tube structure is arranged adjacent the second flexible tube structure, and is arrangeable in a second configuration in which the first flexible tube structure is arranged spaced apart from the second flexible tube structure.

The medicament tube arrangement can thus provide for a reconfiguration providing different arrangements of the first and second flexible tube structure. Hereby, the availability and/or reachability of the flexible tube structures can be varied as the connector enables the first flexible tube structure to be arranged adjacent the second flexible tube structure in its first configuration, and enables the first flexible tube structure to be arranged spaced apart from the second flexible tube structure in its second configuration. Thus, depending on the configuration of the connector, the distance between the first and second flexible tube structures may be varied. Thus, the medicament tube arrangement may suit a wide range of patient anthropometrics, minimizes excess tubing length, and/or may be reliably dispensed to/by the patient. The risk of tube tangling is also decreased. That the connector enables the first flexible tube structure to be arranged adjacent the second flexible tube structure typically implies an efficient and space-saving arrangement of the first and second flexible tube structures, and that the connector enables the first flexible tube structure to be arranged space apart from the second flexible tube structure typically implies that the first flexible tube structure can be moved further away from the second flexible tube structure, and e.g. improve the reachability of the flexible tube structures.

For example, if the patient, or a caregiver, desires to adapt medicament tube arrangement, he/she may interact with the connector and change the configuration of the connector from its first configuration to the second configuration or vice versa. Thus, the connector may be configured to be manually interacted with.

In the present disclosure, the term “longitudinal”, “longitudinally”, “axially” or “axial” refer to a direction extending from a first end to a second end, typically along the device, arrangement or components thereof in the direction of the longest extension of the device, arrangement and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device, arrangement and/or component.

Similarly, “radial” or “radially” refer to a direction extending radially relative to the axis, and “rotation”, “rotational” and “rotationally” refer to rotation relative to the axis.

According to one embodiment, the first flexible tube structure is arranged at a predetermined distance from the second flexible tube structure, wherein the predetermined distance is zero, or close to zero, when the connector is arranged in its first configuration, and wherein the predetermined distance is greater than zero when the connector is arranged in its second configuration, and is at least greater when the connector is arranged in its second configuration as compared to in the first configuration. The predetermined distance is e.g. measured as the shortest distance between the first flexible tube structure and the second flexible tube structure.

According to one embodiment, the connector is configured to extend the radial reachability of medicament tube arrangement. For example, the connector may be configured to arrange a first flexible tube comprising the first flexible tube structure side-by-side with a second flexible tube comprising the second flexible tube structure, i.e. so that the radial extension of the combined first and second flexible tubes increases. For example, the connector may be arranged to, in its first position, connect the first flexible tube radially to the second flexible tube. Moreover, in the second configuration of the connector, the first flexible tube may be spaced apart radially from, e.g. by being moved radially away from, the second flexible tube, e.g. in order to reach different infusion sites. Hereby, the radial reachability of the first and second flexible tubes is increased.

According to one embodiment, at least a part of the connector is configured to be in contact with the first flexible tube structure and the second flexible tube structure in both the first and second configurations. For example, in the first configuration of the connector, a first part of the connector is in contact with the first flexible tube structure and the second flexible tube structure, e.g. to connect the first flexible tube structure to the second flexible tube structure, and a second part of the connector is in contact with only the first flexible tube structure or the second flexible tube structure, whereas in the second configuration of the connector, the first part of the connector is only in contact with the first flexible tube structure or the second flexible tube structure, and the second part of the connector is in contact with the first flexible tube structure and the second flexible tube structure, e.g. to connect the first flexible tube structure to the second flexible tube structure. According to another example, the connector comprises at least two connector portions, wherein a first connector portion is arranged in its first configuration and a second connector portion is arranged in its second configuration. Thus, the connector comprising the first and second connector portions may be arrangeable in the first and second configurations simultaneously.

According to one embodiment, the connector is configured to extend the axial reachability of medicament tube arrangement. That is, the connector may be configured to arrange the first flexible tube structure axially to the second flexible tube structure. In the second configuration of the connector, the first flexible tube structure may be spaced apart axially from, e.g. by being moved axially away from, the second flexible tube structure. Hereby, the axial reachability of the first and second flexible tube structure is increased. Thus, the connector is configured to, at least in the second configuration, extend the axial reachability of medicament tube arrangement.

According to one embodiment, the medicament tube arrangement further comprises: a first flexible tube forming the first flexible tube structure for delivering medicament from a first medicament reservoir, the first flexible tube having a first tube lumen configured to transport the medicament from the first reservoir; a first tube locking protrusion forming the connector, the first tube locking protrusion forming a radial protrusion of the first flexible tube; a second flexible tube forming the second flexible tube structure for delivering medicament from a second medicament reservoir, the second flexible tube having a second tube lumen configured to transport the medicament from the second reservoir and a second tube receiving recess configured to releasably lock to the first tube locking protrusion to co-route the first flexible tube with the second flexible tube.

Thus, in the first configuration of the connector, the first flexible tube, or at least a portion of the first flexible tube, is connected to the second flexible tube, or at least a portion of the second flexible tube. That is, the connector, or the first tube locking protrusion, is releasable locked to the second tube receiving recess in order to connect the first flexible tube to the second flexible tube and arranged the first flexible tube radially adjacent the second flexible tube. Thus, the first configuration of the connector may here be represented by that the connector is received in the second tube receiving recess. The medicament tube arrangement can thus provide for a configuration in which the first flexible tube is efficiently co-routed with the second flexible tube. Hereby, an extensible medicament tube arrangement is provided. Thus, the medicament tube arrangement may suit a wide range of patient anthropometrics, minimize excess tubing length, and may be reliably dispensed to/by the patient. The risk of tube tangling is also decreased.

It should be understood that the first tube lumen is formed in the interior of the first flexible tube, such that a radial cross section of the first flexible tube comprises a cavity or hole representing the first tube lumen. Correspondingly, the second tube lumen is formed in the interior of the second flexible tube, such that a radial cross section of the second flexible tube comprises a cavity or hole representing the second tube lumen.

According to one embodiment, the first flexible tube has a tube length arranged to extend from the first medicament reservoir to a first injection or infusion site, wherein the first tube locking protrusion extends along at least a majority of the tube length of the first flexible tube. Thus, the first flexible tube may be realisably connected to the second flexible tube at least a majority of the tube length of the first flexible tube. That is, the first tube locking protrusion may be provided down the tube length of the first flexible tube. For example, the first flexible tube may be described as extending along a centre axis, and thus, the first tube locking protrusion described as extending in the direction of the centre axis along at least a majority of the tube length.

According to one embodiment, the second flexible tube has a tube length arranged to extend from the second medicament reservoir to a second injection or infusion site, wherein the second tube receiving recess extends along at least a majority of the tube length of the second flexible tube. Thus, the second flexible tube may be realisably connected to the first flexible tube at least a majority of the tube length of the second flexible tube. That is, the second tube receiving recess may be provided down the tube length of the second flexible tube. For example, the second flexible tube may be described as extending along a centre axis, and thus, the second tube receiving recess described as extending in the direction of the centre axis along at least a majority of the tube length.

According to one embodiment, the first tube locking protrusion and the second tube receiving recess are shaped to provide comparatively more flexibility in a first direction than in a second direction. For example, the second direction may be along the previously mentioned centre axis of the first flexible tube, and the second direction being perpendicular to the first direction. This may e.g. be achieved by adapting the moment of inertia in the two directions by designing the first tube locking protrusion and the second tube receiving recess predesigned manner, and/or by adapting the geometry of the first tube locking protrusion and the second tube receiving recess. Hereby, easier routing of the first and second flexible tubes through e.g. clothing while allowing the structure to remain relatively flat may be provided.

According to one embodiment, the tube length of the first flexible tube is different to the tube length of the second flexible tube. Hereby, the first flexible tube and the second flexible tube may be arranged to reach injection or infusion sites at different locations of the user. That is, the medicament tube arrangement may be used for more applications and/or suit a wider range of patient anthropometrics. For example, the first flexible tube is longer than the second flexible tube, or vice versa.

According to one embodiment, the first tube locking protrusion is divided into sub-portions arranged intermittingly along the tube length of the first flexible tube. Thus, the intermittent sub-portions of the first tube locking protrusion may be separated by tube portions having no first tube locking protrusion. Hereby, the first flexible tube may efficiently be released from the second flexible tube at the tube portions having no first tube locking protrusion, i.e. in the spacing between the sub-portions of the first tube locking protrusion. For example, the first flexible tube may be releasably locked to, and co-routed with, the second flexible tube along a first set of sub-portions of the first tube locking protrusion by interaction with the corresponding second tube receiving recess (which may, or may not, be divided into sub-portions arranged intermittingly along the tube length of the second flexible tube), and released from, and hence spaced apart from, the second flexible tube along a second set of sub-portions of the first tube locking protrusion.

According to one embodiment, the medicament tube arrangement is configured to enable that along a first portion of the medicament tube arrangement, the first tube locking protrusion is locked to the second tube receiving recess such that a first portion of the first flexible tube is co-routed with a corresponding first portion of the second flexible tube. In other words, the first flexible tube may be stacked with the second flexible tube along the first portion of the tube arrangement.

According to one embodiment, the medicament tube arrangement is configured to enable that along a second portion of the medicament tube arrangement, the first tube locking protrusion is distant from the second tube receiving recess such that a second portion of the first flexible tube is freely arrangeable in relation to a corresponding second portion the second flexible tube. Hereby, the first flexible tube and the second flexible tube may more easily reach different infusion sites.

According to one embodiment, the medicament tube arrangement further comprises a lock-and-release unit having a main body movable along the first and second flexible tubes in a forward direction and a backward direction, the main body having a first through-hole through which the first flexible tube extends and a second through-hole through which the second flexible tube extends, wherein the first and second through-holes are separated by a spacing, and wherein the spacing is sized such that the first tube locking protrusion of the first flexible tube locks to the second tube receiving recess of the second flexible tube when the main body is moved in the forward direction, and such that the first tube locking protrusion of the first flexible tube is released from the second tube receiving recess of the second flexible tube when the main body is moved in the backward direction. Hereby, the first flexible tube may be efficiently locked and released to and from the second flexible tube. Thus, the previously mentioned first portion of the tube arrangement in which the first tube locking protrusion is locked to the second tube receiving recess may be varied by moving the lock-and-release unit in the forward and backward directions. The forward and backward directions are typically along, or parallel to, the previously mentioned centre axis of the first flexible tube.

According to one embodiment, the medicament tube arrangement further comprises a third flexible tube for delivering medicament from a third medicament reservoir, the third flexible tube having a third tube lumen configured transport the medicament from the third reservoir, and a third tube locking protrusion and/or a third tube receiving recess. Hereby, a third flexible tube may interact with the first flexible tube and/or the second flexible tube in a corresponding manner as the previously described interaction between the first and second flexible tubes.

According to one embodiment, the first tube locking protrusion is a first locking protrusion and the first flexible tube comprises a second locking protrusion configured to releasably lock to the third tube receiving recess. Thus, the first flexible tube may releasably lock to the third flexible tube in a corresponding manner as the previously described interaction between the first and second flexible tubes. For example, the first flexible tube is a centrally arranged flexible tube releasably connected to both the second and third flexible tubes. For example, the second and third flexible tubes are arranged radially of the first flexible tube, but at different directions. For example, the second and third flexible tubes are separated by an angle greater than zero.

According to one embodiment, the second tube receiving recess is a first receiving recess and the second flexible tube comprises a second receiving recess configured to releasably lock to the third tube locking protrusion. Hereby, a corresponding centrally arranged flexible tube may be provided, but where the second flexible tube is the centrally arranged flexible tube.

According to one embodiment, the first flexible tube comprises a first tube receiving recess configured to releasably lock to the third tube locking protrusion of the third flexible tube. Thus, the first flexible tube may releasably lock to the third flexible tube in a corresponding manner as the previously described interaction between the first and second flexible tubes. For example, the first, second and third flexible tubes may be arranged in a stack, at least along said first portion of the medicament tube arrangement, in which the first flexible tube is arranged in between the second flexible tube and the third flexible tube.

According to one embodiment, the medicament tube arrangement further comprises a fourth flexible tube for delivering medicament from a fourth medicament reservoir, the fourth flexible tube having a fourth tube lumen configured transport the medicament from the fourth reservoir, and a fourth tube receiving recess, wherein the second flexible tube comprises a second tube locking protrusion configured to releasably lock to the fourth receiving recess. Thus, the fourth flexible tube may releasably lock to the second flexible tube in a corresponding manner as the previously described interaction between the first and second flexible tubes. For example, the first, second, third and fourth flexible tubes may be arranged in a stack, at least along said first portion of the medicament tube arrangement, in which the first and second flexible tubes are arranged in between the third flexible tube and the fourth flexible tube. Naturally, more than four flexible tubes may be connected in corresponding manner.

According to one embodiment, a radial cross section anywhere along the first flexible tube is non-circular. For example, the radial cross section may be rectangular or square-like with the addition of the first tube locking protrusion (or any locking protrusion) and possibly the first tube receiving recess. Owing to the first tube lumen and the first tube locking protrusion, the radial cross section is typically larger than a corresponding flexible tube having the same size of the tube lumen and e.g. having a circular cross section. A larger cross section typically implies that the first flexible tube is less prone to kinking/occlusion. Additionally, owing to the larger cross section, the first flexible tube is easier to handle by a user without fine motor movement, and easier to route through or around clothing. Moreover, as the first flexible tube is releasably locked to, and co-routed with, the second flexible tube as previously described, the sum of the radial cross sections increases further which further improves the advantages.

Correspondingly for the second flexible tube, a radial cross section anywhere along the first flexible tube is according to one embodiment non-circular. For example, the radial cross section may be rectangular or square-like with the addition of the second tube receiving recess (or any receiving recess) and possibly the first tube locking protrusion.

According to one embodiment, the first flexible tube and the second flexible tube are extruded. Hereby, an efficient way of manufacturing the first flexible tube and the second flexible tube is provided. Thus, the first flexible tube with at least the first tube lumen and the first tube locking protrusion is an extruded flexible tube. Correspondingly, the second flexible tube with at least the second tube lumen and the second tube receiving recess is an extruded flexible tube.

According to one embodiment, inner walls of the first tube lumen is coated with a polymer material. Hereby, the material intended to be in contact with the medicament from the first medicament reservoir may be adapted or chosen accordingly. Correspondingly for the second flexible tube, inner walls of the second tube lumen may according to one embodiment be coated with a polymer material.

According to one embodiment, the first flexible tube with the polymer material of the first tube lumen has been coextruded with the remaining parts of the first flexible tube. Thus, an efficient means for manufacturing the first flexible tube with the polymer material of the first tube lumen is provided. Typically, the remaining parts of the first flexible tube (i.e. not the inner walls of the first tube lumen) is made of a material being different to the polymer material of the first tube lumen.

According to one embodiment, the first lumen is defined by first lumen inner walls, wherein the first lumen inner walls is arranged to be in direct contact with the medicament delivered from the first medicament reservoir. Correspondingly, and according to one embodiment, the second lumen is defined by second lumen inner walls, wherein the second lumen inner walls is arranged to be in direct contact with the medicament delivered from the second medicament reservoir. Thus, the first tube lumen inner walls are forming a portion of, not a separate part to, the first flexible tube. Correspondingly, the first tube locking protrusion is forming a portion of, not a separate part to, the first flexible tube. Correspondingly, and according to one embodiment, the second tube lumen inner walls are forming a portion of, not a separate part to, the second flexible tube. Correspondingly, the second tube receiving recess is forming a portion of, not a separate part to, the second flexible tube. For example, the first tube lumen inner walls may be made in one piece with the first tube locking protrusion and/or, the second tube lumen inner walls may be made in one piece with the second tube receiving recess.

According to one embodiment, the medicament tube arrangement comprises: a flexible tube for delivering medicament, the flexible tube comprising the first flexible tube structure and the second flexible tube structure, the flexible tube having a tube length arranged to extend between the medicament reservoir and an injection or infusion site, and a tube extension section dividing the flexible tube into at least an upstream tube section arranged to extend between the medicament reservoir and the tube extension section, and a downstream tube section arranged to extend between the tube extension section and the infusion or injection site, wherein the connector is arranged in the tube extension section, the connector being configured to, upon being subject to a pulling force larger than a threshold value, re-configure the tube extension section and thereby extend the reachability of the upstream tube section and/or the downstream tube section.

Thus, in the first configuration of the connector, the first flexible tube structure of the flexible tube is connected to the second flexible tube structure of the same flexible tube. Typically, the first flexible tube structure is axially separated from the second flexible tube structure. Thus, in the first configuration of the connector, the first flexible tube structure is arranged adjacent to the second flexible tube structure, i.e. a first portion of the flexible tube is arranged adjacent to a second portion of the flexible tube in order to connect two axially separated portions of the flexible tube. Correspondingly, in the second configuration of the connector, the first flexible tube structure is arranged spaced apart from the second flexible tube structure, i.e. the first portion of the flexible tube is no longer arranged adjacent to the second portion of the flexible tube so that the two axially separated portions of the flexible tube is no longer connected by the connector, or is at least further spaced apart by the connector as compared to when the connector is arranged in its first configuration. Hereby the reachability, or axial reachability, may be extended. The connecter is thus configured to, upon being subject to a pulling force larger than a threshold value, re-configure the tube extension section by changing the configuration of the connector from its first configuration to its second configuration. In other words, the extended reachability is typically referring to the axial reachability of the upstream tube section and/or the downstream tube section.

Thus, instead of the previous mentioned embodiments in which the first flexible tube structure is comprised in, or composed of, the first flexible tube and the second flexible tube structure is comprised in, or composed of, the second flexible tube, the first and second flexible tube structure is here comprised in a single, or common, flexible tube. Thus, throughout the application text, when referring to the flexible tube (or a flexible tube) with the prefix “first”, “second” or the like, such flexible tube may be referred to as the single, or common, flexible tube.

The axial reachability is thus referring to the reachability of the upstream tube section and/or the downstream tube section in a direction from the medicament reservoir to the injection or infusion site (i.e. along an axis of the medicament tube arrangement, or infusion set, extending from the medicament reservoir to the injection or infusion site, e.g. extending straight from the medicament reservoir to the injection or infusion site). Correspondingly, the radial reachability may be defined in a direction extending radially to such axis, or to a centre axis of the first and/or second flexible tube.

According to one embodiment, the connector comprises a first locking protrusion and/or a first receiving recess. According to one embodiment, the first flexible tube structure and/or the second flexible tube structure comprises a corresponding second locking protrusion being configured to releasably lock to the first receiving recess and/or a corresponding second receiving recess being configured to releasably lock to the first locking protrusion. Hereby, the connector may be releasably locked to the first flexible tube structure and/or the second flexible tube structure. In other words, upon being subject to a pulling force larger than a threshold value, the connector may be released from the first flexible tube structure and/or the second flexible tube structure. That is, the connector may be brought into its second configuration by that the first locking protrusion is released from the second receiving recess, and/or by that the second locking protrusion is released from the first receiving recess. Thereafter, the connector may be brought into its first configuration again by releasably locking the first locking protrusion to the second receiving recess, and/or by releasably locking the second locking protrusion to the first receiving recess. Thus, the configuration of the tube extension section and the reachability of the upstream tube section and/or the downstream tube section may be adapted by the releasably locking of the first locking protrusion and the second receiving recess and/or the second locking protrusion and the first receiving recess. For example, the connector may be fixedly connected to the first flexible tube structure at one end, and at an opposite end comprising the first locking protrusion, wherein the second flexible tube structure comprises the second receiving recess configured to releasably lock to the first locking protrusion. Typically, the features mentioned in relation to the first tube locking protrusion are applicable to the first or second locking protrusions, and the features mentioned in relation to the second tube receiving recess are applicable are applicable to the first or second receiving recess.

The medicament tube arrangement can thus provide for a reconfiguration of the tube extension section and thereby increase the availability of the upstream tube section and/or the downstream tube section. Hereby, an extensible medicament tube arrangement is provided. Thus, the medicament tube arrangement may suit a wide range of patient anthropometrics, minimize excess tubing length, and may be reliably dispensed to/by the patient. The risk of tube tangling may also be decreased. That the reachability of the upstream tube section and/or the downstream tube section is extended, typically implies that the upstream tube section can be moved away further from the injection or infusion site, and/or that the downstream tube section can be moved away further from the medicament reservoir.

For example, if the patient desires to adapt, or tamper with, the medicament reservoir, e.g. the connection or interface between the flexible tube and the medicament reservoir, the patient may grip the upstream tube section and apply a pulling force (typically in a direction towards the medicament reservoir) larger than the threshold value in order to reconfigure the tube extension section and thereby increase the reachability, or axial reachability, of the upstream tube section by moving it further away from the injection or infusion site. By applying a pulling force to the upstream tube section, the connector being arranged in the tube extension section will be subjected to the pulling force and thereby reconfigure the tube extension section. Stated differently, by subjecting the upstream tube section, and thereby the connector in the tube extension section, to a pulling force larger than the threshold value, the tube extension section is re-configured and at least a part is moved closer to the medicament reservoir, thereby increasing the reachability, or axial reachability, of the upstream tube section. Correspondingly, if the patient desires to adapt, or tamper with, the injection or infusion site, e.g. a needle delivering medicament, the patient may grip the downstream tube section and apply a pulling force (typically in a direction towards the injection or infusion site) larger than the threshold value in order to reconfigure the tube extension section and thereby increase the reachability, or axial reachability, of the downstream tube section by moving it further away from the medicament reservoir. By applying a pulling force to the downstream tube section, the connector being arranged in the tube extension section will be subjected to the pulling force and thereby reconfigure the tube extension section. Stated differently, by subjecting the downstream tube section, and thereby the connector in the tube extension section, to a pulling force larger than the threshold value, the tube extension section is re-configured and at least a part is moved closer to the injection or infusion site, thereby increasing the reachability, or axial reachability, of the downstream tube section.

That is, owing to the reconfigurable tube extension section, the reachability, or axial reachability, of the upstream tube section and/or the downstream tube section can be extended, or increased, in response to that the connector is subjected to a pulling force larger than the threshold value. The connector may be configured to hold the tube extension section, and upon being subject to the pulling force larger than the threshold value, re-configure the tube extension section by at least partly unfold, or release, the tube extension section.

According to one embodiment, the connector has a main body comprising a first end portion having a first contact surface, and a second end portion arranged opposite to the first end portion, the second end portion having a second contact surface, wherein the first contact surface is attached to a first connecting portion of the flexible tube in the tube extension section, the first connecting portion forming the first flexible tube structure, and the second contact surface is attached to a second connecting portion of the flexible tube in the tube extension section, the second connecting portion forming the second flexible tube structure, wherein the first and second contacting portions of the flexible tube in the tube extension section are distant from each other. Thus, the connector connects two distant portions, i.e. the first and second contacting portions or first and second flexible tube structures, of the flexible tube in the tube extension section. Typically, the length of the flexible tube section between the first and second contacting portions is greater than a length of the main body extending between the first and second end portions. Hereby, the flexible tube section between the first and second contacting portions, which forms at least a part of the tube extension section, is arranged to be held in a space saving manner by the connector.

According to one embodiment, the tube extension section is defined to extend from the first connecting portion to the second connecting portion. That is, the previously mentioned flexible tube section between the first and second contacting portions forms the tube extension section.

According to one embodiment, the connector comprises a first tubular tube holder encompassing the first contacting portion of the flexible tube, the first tubular tube holder comprising the first contact surface, and a second tubular tube holder encompassing the second contacting portion of the flexible tube, the second tubular tube holder comprising the second contact surface. Hereby, the connector may hold the flexible tube and the tube extension section in an efficient manner. For example, the first and second tubular tube holders are configured to firmly hold the corresponding first and second contacting portion of the flexible tube. The first and second tubular tube holders may be configured to hold the corresponding first and second contacting portion of the flexible tube by a friction fit. According to one alternative embodiment, the tube extension section is defined to extend from the first tubular tube holder to the second tubular tube holder. Thus, regardless of the configuration of the tube extension section, e.g. regardless of if the tube extension section is fully protracted (e.g. as a result of that the flexible tube of the tube extension section being straightened to its full length) or not, the downstream tube section may be defined downstream from the first tubular holder and the upstream tube section may be defined upstream from the second tubular holder.

According to one embodiment, the first connecting portion and the second connecting portion are separated by a loop of the flexible tube, or a tube bend of the flexible tube. Hereby, the flexible tube may be arranged in a space saving manner. Stated differently, the section of the flexible tube extending between the first and second connecting portions may be folded or bended, and be held it its folded or bended configuration by the connector. Thus, as the connector is subjected to a pulling force larger than a threshold value, the tube extension section is re-configured by being at least partly unfolded or unbended (e.g. untying the loop or straightening of the tube bend) and thereby extend the reachability of the upstream tube section and/or the downstream tube section. Thus, the tube extension section may be arrangeable in a folded or bended configuration in which the section of the flexible tube extending between the first and second connecting portions is folded, e.g. as a loop, or bended, e.g. as a tube bend, and arrangeable in an unfolded or unbended configuration, or at least a partly unfolded or unbended configuration as compared to the folded or bended configuration, in which the section of the flexible tube extending between the first and second connecting portions is at least partly unfolded, or unended (i.e. straightened), e.g. as an at least partly untied loop, or at least partly straightened tube bend.

According to one embodiment, the medicament tube arrangement is arrangeable in a contracted condition, in which the tube extension section is arranged in the folded or bended configuration, and in a protracted condition, in which the tube extension section is arranged in the unfolded or unbended configuration. In the protracted condition, the reachability of the upstream tube section and/or the downstream tube section is increased as compared to in the contracted condition. In other words, the effective length of the flexible tube is increased in the protracted condition as compared to in the contracted condition.

According to one embodiment, the main body of the connector is stretchable. Hereby, the tube extension section may be reconfigured by stretching the main body of the connector. For example, upon being subjected to a pulling force larger than a threshold value, the main body of the connector is stretched, to thereby reconfigure the tube extension section to at least partly unfold, or be at least partly straightened. As previously mentioned, this extends the reachability of the upstream tube section and/or the downstream tube section. According to one embodiment, the main body of the connector is arrangeable in a relaxed state, e.g. an un stretched state, in which the tube extension section is arranged in its folded or bended configuration, and in an unrelaxed state, e.g. a stretched, or tensioned state, upon being subjected to a pulling force larger than a threshold value. In the unrelaxed state, the spacing between the first and second contact surfaces of the connector is larger than in the relaxed state. According to one embodiment, at least a part of the main body of the connector is stretchable. For example, and with reference to the embodiment comprising the first and second tubular tube holders, an interconnecting portion extending from the first tubular tube holder to the second tubular tube holder is stretchable.

According to one embodiment, the main body is strain hardening such that the main body is extended progressively. Hereby, a varying extension profile of the main body of the connector is provided. Thus, the tube extension section may be unfolded or unbended gradually, or in discrete steps.

According to one embodiment, the main body of the connector is breakable. By breakage of the main body of the connector, the tube extension section may be completely unfolded or straightened. That is, the effective full length of the tube extension section may be made accessible.

According to one embodiment, the main body is breakable upon being subject to a pulling force larger than the threshold value. That is, upon subjecting the connector to a pulling force larger than the threshold value, the main body of the connector is configured to break.

It should be understood that the embodiments in which the main body is stretchable, or is strain hardening such that the main body is extended progressively, may be combined with the embodiment in which the main body is breakable. For example, the main body of the connector may be configured to, upon being subject to a pulling force larger than a first threshold value, stretch or extend (e.g. in a discrete step to a predetermined length of the main body), and upon being subject to a pulling force larger than a second threshold value, the second threshold value being greater than the first threshold value, break. In other words, the main body of the connector may be configured to extend, or progressively extend, until a fracture stress, at which point the main body will behave like the frangible section.

According to one embodiment, the main body of the connector is elongated. Thus, a sufficient spacing between the first and second contact surfaces may be provided. Moreover, by adapting the size of the elongated main body, i.e. the length of the elongated main body, the connector may be adapted for different types of tube extension sections.

According to one embodiment, the dead space of the flexible tube is the same regardless of the configuration of the tube extension section. Hereby, the flexible tube is configured to have a constant volume of retained medication regardless of the configuration of the tube extension section. Thus, there is no need to vary the medicament volume above a nominal volume for delivering a nominal dose of medicament to the patient. Stated differently, by providing a constant dead space regardless of the configuration of the tube extension section, a predictable dose of medicament can be delivered to the patient. In other words, the tube length of the flexible tube is the same regardless of the configuration of the tube extension section, only the reachability of the upstream tube section and/or the downstream tube section varies with the configuration of the tube extension section. In other words, the effective tube length of the flexible tube may be described as to vary with the configuration of the tube extension section.

According to one embodiment, the connector is made of a polymer material. According to one embodiment, at least the main body of the connector is made of a polymer material. The chosen polymer material may be stretchable, or strain hardening, in correspondence with the earlier described embodiments. The chosen polymer material may be breakable upon being subject to a pulling force larger than the threshold value, in correspondence with the earlier described embodiments. That is, by choosing the polymer material of the connector, or at least of the main body of the connector, desirable characteristics of the connector, such that the main body is stretchable or strain hardening and/or breakable, may be easily achieved. The polymer material may be a thermoplastic elastomer, polypropylene, or other suitable polymer know to the skilled person.

According to one embodiment, the is made of a polymer material providing elastic hysteresis. Elastic hysteresis means that there is a difference between the strain energy required to generate a given stress in the material, and the material's elastic energy at that stress. Hereby, desirable characteristics of the connector may be achieved.

According to one embodiment, the tube extension section comprises a plurality of tube extension sub-sections arranged along the flexible tube, and wherein the at least one connector is a plurality of connectors, each connector being associated with a corresponding tube extension sub-section. Herby, the medicament tube arrangement is improved, e.g. the space saving arrangement of the flexible tube is improved. For example, the reachability of upstream tube section and/or the downstream tube section can be extended or increased in discrete steps, corresponding to the plurality of tube extension sub-sections.

According to one embodiment, the connectors in the plurality of connectors are correspondingly arranged and configured, e.g. by being designed and constructed equally, e.g. according to any one of the previously described embodiments. For example, the stretchability, or breakability, of the main bodies may be the same for each connector. According to one embodiment, the tube extension sub-sections in the plurality of tube extension sub-sections are correspondingly arranged and configured, e.g. by being reconfigurable equally, e.g. according to any one of the previously described embodiments. For example, the amount, or length, of flexible tube may be the same in each one of the tube extension sub-sections.

According to one embodiment, at least two of the connectors in the plurality of connectors are arranged and configured differently, e.g. by being designed and constructed differently, e.g. according to at least two different embodiments of the previously described embodiments. For example, the stretchability, or breakability, of the main bodies may be different for at least two connectors. According to one embodiment, at least two of the tube extension sub-sections in the plurality of tube extension sub-sections are arranged and configured differently, e.g. by being reconfigurable differently, e.g. according to at least two different embodiment of the previously described embodiments. For example, the amount, or length, of flexible tube may be different between at least two tube extension sub-sections.

According to one embodiment, each one of the connectors has a main body comprising a first end portion having a first contact surface, and a second end portion arranged opposite to the first end portion, the second end portion having a second contact surface, wherein the first contact surface of each main body is attached to a corresponding first connecting portion of the flexible tube in a corresponding tube extension sub-section, and the second contact surface of each main body is attached to a corresponding second connecting portion of the flexible tube in the corresponding tube extension sub-section, wherein the first and second contacting portions of the flexible tube in each one of the tube extension sub-sections are distant from each other. Thus, each connector connects two distant portions, i.e. the first and second contacting portions, of the flexible tube in each one of the tube extension sub-sections. Typically, the length of the flexible tube section between the first and second contacting portions in each of the tube extension sub-sections is greater than a length of the corresponding main body extending between the corresponding first and second end portions. Hereby, the flexible tube section between the corresponding first and second contacting portions in each one of the tube extension sub-sections, which forms a part of the tube extension section, is arranged to be held in a space saving manner by the corresponding connector. Each tube extension sub-section may be defined to extend from the corresponding first connecting portion to the corresponding second connecting portion.

According to one embodiment, a first tube extension sub-section is arranged furthest downstream of the plurality of tube extension sub-sections, and a last tube extension sub-section is arranged furthest upstream of the plurality of tube extension sub-sections. No, one or more tube extension sub-sections may be arranged in between the first tube extension sub-section and the last tube extension sub section. Thus, the tube extension section may be defined to extend from the first connecting portion of the first tube extension sub-section to the second connecting portion of the last tube extension sub-section. That is, the upstream tube section is arranged upstream of the last tube extension sub-section, and the downstream tube section is arranged downstream of the first tube extension sub-section.

According to one embodiment, each connector comprises a main body being breakable upon being subject to a pulling force larger than a threshold value, and wherein the threshold value varies for the different main bodies of the connectors. Thus, each connector in the plurality of connectors comprises main body as previously described, wherein each one of the main bodies brakes at different pulling forces (i.e. they have different breaking forces). For example, the breaking force of each contactor may gradually increase from the first tube extension sub-section to the last tube extension sub-section (or vice versa) so that the flexible tube unwinds or unfolds from upstream to downstream (or “top to bottom”), or downstream to upstream (or “bottom to top”).

The previously mentioned threshold value of the pulling force may according to one embodiment be set to be smaller than the required force need to remove the needle from the infusion or injection site, e.g. related to the force needed to remove a needle adhesive. Hereby, the reachability of the upstream tube section and/or the downstream tube section may be extended without affecting the needle at the infusion or injection site.

According to one embodiment, the medicament tube arrangement further comprises a housing arranged to cover the tube extension section. Hereby, the tube extension section may be efficiently stored in the housing. For the previously mentioned embodiment having a plurality of tube extension sub-sections, each one of the plurality of tube extension sub-sections is preferably arranged inside the housing. For example, the housing is arranged to cover the tube extension section, or the plurality of tube extension sub-sections, at least in the previously mentioned retracted state of the medicament tube arrangement.

According to one embodiment, the housing comprises a first opening, wherein the downstream tube section extends through the first opening such that a predefined length of the flexible tube is available outside of the housing. That is, the predefined length of the flexible tube is available outside of the housing downstream of the first opening. The predefined length may be adapted to at least provide a sufficient gripping portion of the downstream tube section, such that the patient may grip the downstream tube section and pull the flexible tube, e.g. in a direction towards the injection or infusion site, to induce a pulling force to the contactor larger than the threshold value. The housing may be made of a flexible material, or a rigid material. The portion of the flexible tube extending through the first opening of the housing and having the predefined length may be provided with information of the medicament tube arrangement and/or the medicament in the medicament reservoir, for example in the form of a barcode (e.g. a QR code) or other digital readable marker.

According to one embodiment, the housing comprises a second opening arranged distant to the first opening (e.g. opposite to the first opening), wherein the upstream tube section extends through the second opening such that a predefined length of the flexible tube is available outside of the housing upstream of the second opening. The predefined length of the flexible tube available outside of the housing upstream of the second opening may be used in a corresponding manner as the predefined length of the flexible tube available outside of the housing downstream of the first opening.

According to one alternative embodiment, the connector is breakable upon being subjected to a pulling force larger than the threshold value, and is configured to eliminate the tube extension section (or at least one of the first and last tube extension sub-sections) upon breaking. For example, by eliminating the last tube extension sub-section, the previous second last tube extension sub-section is redefined as the new last tube extension sub-section, and the length of the upstream tube section is increased. Correspondingly, by eliminating the first tube extension sub-section, the previous second first tube extension sub-section (i.e. a second tube extension sub-section) is redefined as the new first tube extension sub-section, and the length of the downstream tube section is increased.

There is according to a second aspect provided an infusion set comprising a medicament delivery device and a medicament tube arrangement according to the first aspect.

Effects and features of the second aspect are largely analogous to those described above in connection with the first aspect. Embodiments mentioned in relation to the first aspect are largely compatible with the second aspect.

According to one embodiment, the tube length of the flexible tube extends between the medicament reservoir and the injection or infusion site. According to one embodiment, the upstream tube section extends between the medicament reservoir and the tube extension section. According to one embodiment, the downstream tube section extends between the tube extension section and the infusion or injection site. According to one embodiment, applicable to any one of the first and second aspects, the infusion set and/or the medicament tube arrangement for the infusion set comprises the medicament reservoir. According to one embodiment, applicable to any one of the first and second aspects, the infusion set and/or the medicament tube arrangement for the infusion set comprises an injection assembly for medicament administration at the injection or infusion site. According to one embodiment, applicable to any one of the first and second aspects, the tube length of the flexible tube extends between the medicament reservoir and the injection assembly. According to one embodiment, applicable to any one of the first and second aspects, the upstream tube section extends between the medicament reservoir and the tube extension section. According to one embodiment, applicable to any one of the first and second aspects, the downstream tube section extend between the tube extension section and the injection assembly.

According to one embodiment, applicable to any one of the first and second aspects, the infusion set and/or the medicament tube arrangement for the infusion set, further comprises an injection assembly for medicament administration comprising a first needle fluidly connected to a first medicament reservoir via a first flexible tube for a first predefined medicament administration, and a second needle fluidly connected to a second medicament reservoir via a second flexible tube for a second predefined medicament administration. Each one of the first and second flexible tubes may be arranged as described with reference to the first aspect. Thus, the infusion set and/or the medicament tube arrangement for the infusion set may provide multiple medicament administrations, e.g. multiple (and possibly different) medications. Each one of the first and second needles may for example be one of a right angle needle, a straight needle, or a soft cannula.

According to one embodiment, applicable to any one of the first and second aspects, the infusion set and/or the medicament tube arrangement for the infusion set comprises the previously described first medicament reservoir. According to one embodiment, applicable to any one of the first and second aspects, the infusion set and/or the medicament tube arrangement for the infusion set comprises a first injection assembly for medicament administration at the first injection or infusion site. According to one embodiment, applicable to any one of the first and second aspects, the tube length of the first flexible tube extends from the first medicament reservoir to the first injection assembly. Correspondingly, and according to one embodiment, applicable to any one of the first and second aspects, the infusion set and/or the medicament tube arrangement for the infusion set comprises the second medicament reservoir. According to one embodiment, applicable to any one of the first and second aspects, the infusion set and/or the medicament tube arrangement for the infusion set comprises a second injection assembly for medicament administration at the second injection or infusion site. According to one embodiment, applicable to any one of the first and second aspects, the tube length of the second flexible tube extends from the second medicament reservoir to the second injection assembly.

With predefined medicament administration is meant that a predefined type of drug/medicament is to be administered at a predefined rate, for a predefined amount of time. The target patient group may also be predefined.

Generally, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 shows an example of a medicament tube arrangement, and an infusion set comprising such medicament tube arrangement;

FIGS. 2A-2B show an enlarged view of a part of a flexible tube and a connector in a tube extension section, or a tube extension sub-section according to at least one example embodiment;

FIG. 3 show an enlarged view of a part of a flexible tube and a connector in a tube extension section, or a tube extension sub-section according to at least one example embodiment;

FIG. 4 show an enlarged view of a part of a flexible tube and a connector in a tube extension section, or a tube extension sub-section according to at least one example embodiment;

FIG. 5 show an enlarged view of a part of a flexible tube and a connector in a tube extension section, or a tube extension sub-section according to at least one example embodiment;

FIG. 6 shows an example of a medicament tube arrangement, and an infusion set comprising such medicament tube arrangement;

FIGS. 7A and 7B show the medicament tube arrangement of FIG. 1 further comprising a housing arranged to cover the tube extension section, and a detailed view of the flexible tube available outside of the housing,

FIG. 8 shows an example of a medicament tube arrangement, and an infusion set comprising such medicament tube arrangement;

FIGS. 9A and 9B are radial cross sectional views of the first flexible tube and the second flexible tube of the medicament tube arrangement of FIG. 8;

FIG. 10 is a cross sectional side view of an example embodiment of a lock-and-release unit of a medicament tube arrangement;

FIG. 11 shows an example of medicament tube arrangement for an infusion set;

FIGS. 12A and 12B are radial cross sectional views of an example embodiment of the first flexible tube, the second flexible tube and the third flexible tube of the medicament tube arrangement of FIG. 8;

FIG. 13 shows a cross sectional view of another example of a medicament tube arrangement;

FIG. 14 shows a perspective view of the first flexible tube of the embodiment of FIG. 8;

FIG. 15 shows a perspective view of the second flexible tube of the embodiment of FIG. 8;

FIG. 16 shows a perspective view of an example embodiment of a first flexible tube; and

FIG. 17 shows a radial cross section of an example embodiment of a first flexible tube.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the example embodiments set forth herein; rather, these example embodiments are provided by way of example so that this disclosure will be thorough and complete, and will fully convey the scope of the inventive concept to those skilled in the art. Like numbers refer to like elements throughout the description.

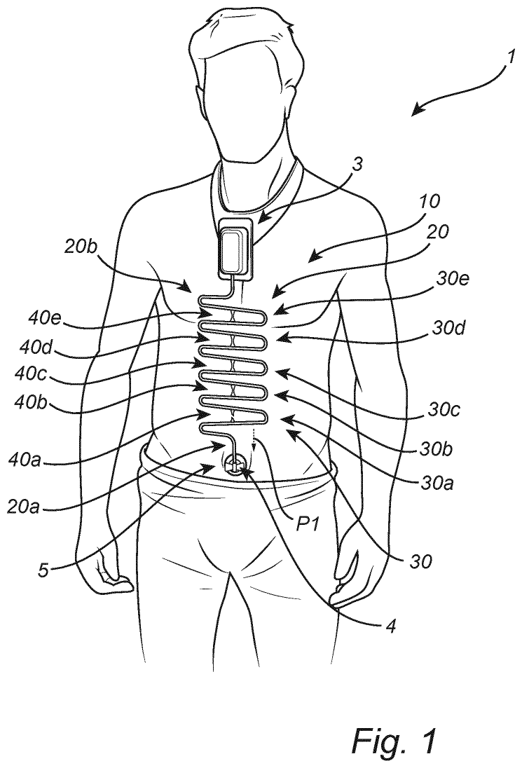

FIG. 1 shows a first example of medicament tube arrangement 10, and an infusion set 1 comprising such medicament tube arrangement 10 according to an example embodiment. The infusion set 1 comprises a medicament reservoir 3, a needle 4 for delivering medicament at an injection or infusion site 5, and the medicament tube arrangement 10 arranged in between the medicament reservoir 3 and the injection or infusion site 5 (or needle 4). The needle 2 may be attached to the patient at the injector or infusion site 5 by means of a pad using a needle adhesive. The medicament reservoir 3 and the needle 4 may together form, or form parts of, a medicament delivery device.

The medicament tube arrangement 10 comprises a flexible tube 20 for delivering medicament. The flexible tube 20 has a tube length extending between the medicament reservoir 3 and the injection or infusion site 5, as it is arranged to fluidly connect the medicament reservoir 3 with the needle 4. Thus, during use, medicament can be delivered from the medicament reservoir 3 to the needle 4 at the injection or infusion site 5 by being fluidly transported in the flexible tube 20 of the medicament tube arrangement 10. The medicament reservoir 3 may e.g. be equipped with a pressure drive, such as e.g. a pump, for providing the necessary pressurization of the medicament.

As seen in FIG. 1, the flexible tube 20 comprises a tube extension section 30 in which the flexible tube 20 is bundled up, or folded, by a plurality of tube extension sub-sections 30a, 30b, 30c, 30d, 30e. The tube extension section 30 divides the flexible tube 20 into at least an upstream tube section 20b extending between the medicament reservoir 3 and the tube extension section 30, and a downstream tube section 20a extending between the tube extension section 30 and the infusion or injection site 5.

The medicament tube arrangement 10 comprises a plurality of connectors 40a, 40b, 40c, 40d, 40e arranged in the tube extension section 30, of which at least one connector is needed. Each one of the connectors 40a, 40b, 40c, 40d, 40e is configured to, upon being subject to a pulling force P1 larger than a threshold value, re-configure the tube extension section 30 to thereby extend the reachability of the upstream tube section 20b and/or the downstream tube section 20a as will be described in the following.

In more detail, the tube extension section 30 in FIG. 1 comprises a plurality of tube extension sub-sections 30a, 30b, 30c, 30d, 30e, here being a first tube extension sub-section 30a arranged furthest downstream, a last tube extension sub-section 30e arranged furthest upstream, and three intermediate tube extension sub-sections 30b, 30c, 30d arranged in between the first and last tube extension sub-sections 30a, 30e. Each one of the plurality of connectors 40a, 40b, 40c, 40d, 40e is associated with a corresponding tube extension sub-section 30a, 30b, 30c, 30d, 30e, such that e.g. a first connector 40a is arranged the first tube extension sub-section 30a, a last connector 40e is arranged in the last tube extension sub-section 30e, and each one of three intermediate connectors 40b, 40c, 40d arranged in between the first and last connectors 40a, 40e is arranged in an associated intermediate tube extension sub-section 30b, 30c, 30d.

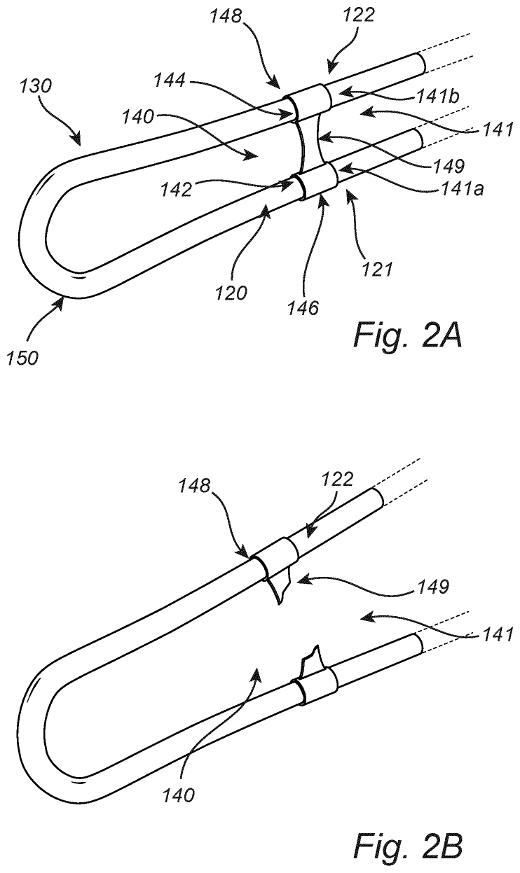

Turning to FIG. 2A showing an enlarged view of a part of a flexible tube 120 and a connector 140 in a tube extension section 130, or a tube extension sub-section according to at least one example embodiment. The flexible tube 120 may be that of FIG. 1, and the connector 140 may any one of the plurality of connectors 40a, 40b, 40c, 40d, 40e arranged in a corresponding tube extension sub-section. Thus, the tube extension section 130 may be any one of the tube extension sub-sections 30a, 30b, 30c, 30d, 30e of FIG. 1. As an alternative, the tube extension section 130 comprises only the one connector 140, and the tube extension section 130 of FIG. 2A may correspond to the tube extension section 30 of FIG. 1.

The connector 140 has a main body 141 comprising a first end portion 141a having a first contact surface 142, and a second end portion 141b arranged opposite to the first end portion 141a, the second end portion 141b having a second contact surface 144. The first contact surface 142 is attached to a first connecting portion 121 of the flexible tube 120 in the tube extension section 130, or tube extension sub-section (shown in FIG. 1). The second contact surface 144 is attached to a second connecting portion 122 of the flexible tube 120 in the tube extension section 130 or tube extension sub-section (shown in FIG. 1). As seen in FIG. 2A, the first and second contacting portions 121, 122 of the flexible tube 120 are distant from each other. Hereby, the tube extension section 130 may be arranged in a space saving manner by the connector 140.

With reference to the previously mentioned embodiment in which the tube extension section 130 is any one of the tube extension sub-sections 30a, 30b, 30c, 30d, 30e of the medicament tube arrangement 10 of FIG. 1, each one of the tube extension sub-sections 30a, 30b, 30c, 30d, 30e may be defined to extend from a corresponding first connecting portion to a corresponding second connecting portion of the flexible tube 20. As an alternative, and with reference to the previously mentioned embodiment in which the tube extension section 130 comprises only the one connector 140, and the tube extension section 130 of FIG. 2A corresponds to the tube extension section 30 of FIG. 1, the tube extension section 130 may be defined to extend from the first connecting portion 121 to the second connecting portion 122 of the flexible tube 120.

In the embodiment of FIG. 2A, the connector 140 comprises a first tubular tube holder 146 encompassing the first contacting portion 121 of the flexible tube 120. The first tubular tube holder 146 comprises the first contact surface 142 of the connector 140. Moreover, a second tubular tube holder 148 encompasses the second contacting portion 122 of the flexible tube 120, the second tubular tube holder 148 comprising the second contact surface 144 of the connector 140. The first tubular tube holder may be connected to the second tubular tube holder by an interconnecting portion 149 of the main body 141. The interconnecting portion 149 is preferably elongated, thereby forming an elongated main body 141 of the connector 140. The first and second tubular tube holders 146, 148 may be configured to firmly hold the corresponding first and second contacting portion 121, 122 of the flexible tube 120.

The tube extension section 130, or any one of the tube extension sub-sections 30a, 30b, 30c, 30d, 30e, may be formed by a tube bend 150 of the flexible tube 20, 120. That is, the first connecting portion 121 and the second connecting portion 122 of the flexible tube 120 may be separated by the tube bend 150. Hereby, the flexible tube 120 may be arranged in a space saving manner.

The main body 141 of the connector 140 may be breakable. For example, the interconnecting portion 149 between the first and second tubular tube holders 146, 148 may be breakable. Thus, upon being subject to a pulling force P1 larger than the threshold value as previously described, the main body 141, or the interconnecting portion 149 of the main body 141, is configured to break. As the main body 141 breaks, as shown in FIG. 2B, the tube extension section 130 is reconfigured. In the example embodiment of FIGS. 2A and 2B (and correspondingly for the tube extension sub-sections of FIG. 1), as the connector 140 is subjected to the pulling force P1 larger than the threshold value, the tube extension section 130 is re-configured as the main body 141 breaks, and thereby the flexible tube 120 in the tube extension section 130 is unbended (i.e. the flexible tube 120 is straightened). Hereby, and with reference to both FIGS. 1 and 2A, the reachability of the upstream tube section 20b and/or the downstream tube section 20a is increased. Stated differently, the tube extension section 30, 130 may be arrangeable in a folded or bended configuration in which the flexible tube 20, 120 is folded, e.g. as one or more tube bends 150 as shown in FIGS. 1 and 2A, and arrangeable in an unfolded or unbended configuration in which the flexible tube 20, 120 is at least partly unfolded, or unended (i.e. straightened). The unfolded or unbended configuration of the tube extension section 30, 130 is achieved by the subjecting one or more of the connectors 40a, 40b, 40c, 40d, 40e, 140 to a pulling force P1 larger than the threshold value.

As seen in FIG. 1, the pulling force P1 may e.g. be in a direction towards the injection or infusion site 5 in order to extend the reachability of the downstream tube section 20a.

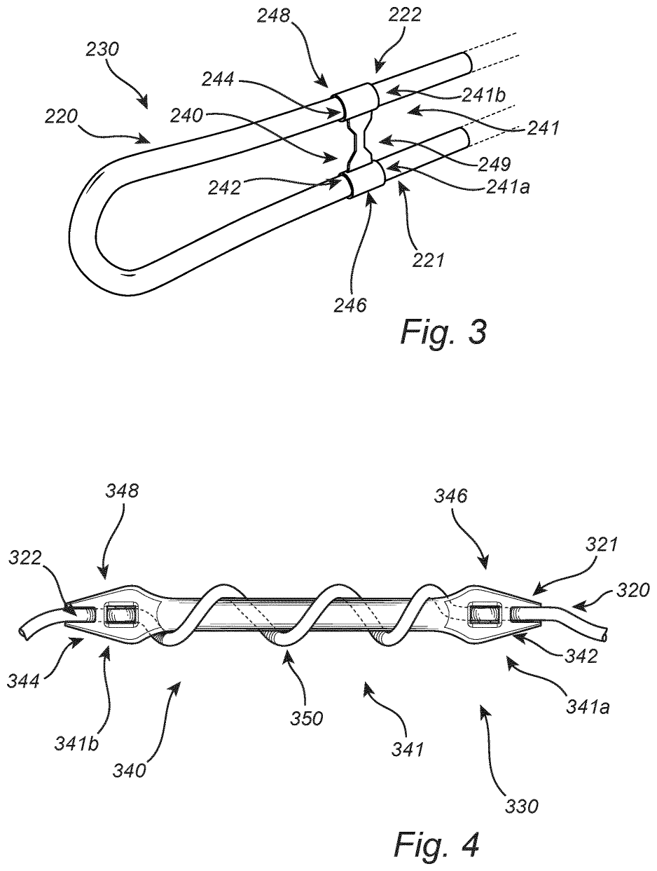

Turning to FIG. 3, showing an enlarged view of a part of a flexible tube 220 and a connector 240 in a tube extension section 230 (or a tube extension sub-section) according to at least one example embodiment. The flexible tube 220 may be that of FIG. 1, and the connector 240 may be any one of the plurality of connectors 40a, 40b, 40c, 40d, 40e arranged in a corresponding tube extension sub-section. Thus, the tube extension section 230 may be any one of the tube extension sub-sections 30a, 30b, 30c, 30d, 30e of FIG. 1. As an alternative, the tube extension section 230 may comprise only the one connector 240, and the tube extension section 230 of FIG. 3 may correspond to the tube extension section 30 of FIG. 1.

As for the embodiment of FIG. 2A, the connector 240 comprises a main body 241, a first end portion 241a having a first contact surface 242, a second end portion 241b having a second contact surface 244, and first and second tubular tube holders 246, 248 encompassing corresponding first and second contacting portions 221, 222 of the flexible tube 220 in the tube extension section 230. As the structure and functions associated with these feature are corresponding those described for the embodiment of FIG. 2A, the features are not described in detail again.

The main body 241 of the connector 240 comprises an interconnecting portion 249 extending between the first and second tubular tube holders 246, 248 as for the embodiment of FIG. 2A, forming an elongated main body 241. However, the interconnecting portion 249 is not necessarily breakable, but stretchable. That is, the main body 141, or at least a portion of the main body 141 such as the interconnecting portion 249, of the connector 140 is stretchable. Thus, the main body 241, or the interconnecting portion 249 of the main body 241, is configured to stretch upon being subjected to the pulling force P1. As the main body 241 stretches, the tube extension section 230 is reconfigured. In the example embodiment of FIG. 3, as the connector 240 is subjected to the pulling force P1 larger than the threshold value, the tube extension section 230 is re-configured as the main body 241 stretches and thereby the flexible tube 220 in the tube extension section 230 is at least partly unbended (i.e. the flexible tube 220 is at least partly straightened). Hereby, the reachability of the upstream tube section and/or the downstream tube section is increased as previously described.

For example, the main body 241, or at least the interconnecting portion 249, may be strain hardening such that the main body 241, or at least the interconnecting portion 249, is extended progressively. Hereby, a varying extension profile of the connector 240 is provided. Thus, the tube extension section 230 may be unfolded or unbended gradually, or in discrete steps. For example, upon being subjected to a pulling force P1 larger than a first threshold value, the main body 241 may stretch or extend in a discrete step to a predetermined first length of the interconnecting portion 249, and upon being subject to a pulling force P1 larger than a second threshold value, the second threshold value being greater than the first threshold value, stretched or extended in another discrete step to a predetermined second length of the interconnecting portion 249, the predetermined second length being larger than the predetermined first length. As an alternative, the main body 241, e.g. at the interconnecting portion 249, may break upon being subject to a pulling force P1 larger than the second threshold value. Thus, embodiments in which the main body 241 is stretchable, or is strain hardening such that the main body 241 is extended progressively, may be combined with embodiments in which the main body 141 is breakable. In other words, the main body 241 of the connector 240 may be configured to extend, or progressively extend, until a fracture stress, at which point the main body 241 will behave like the frangible section, the frangible section being e.g. the interconnecting portion 249.

Turning to FIG. 4, showing an enlarged view of a part of a flexible tube 320 and a connector 340 in a tube extension section 330 (or a tube extension sub-section) according to at least one example embodiment. The flexible tube 320 may be that of FIG. 1, and the connector 340 may any one of the plurality of connectors 40a, 40b, 40c, 40d, 40e arranged in a corresponding tube extension sub-section. Thus, the tube extension section 330 may be any one of the tube extension sub-sections 30a, 30b, 30c, 30d, 30e of FIG. 1. As an alternative, the tube extension section 330 comprises only the one connector 340, and the tube extension section 330 of FIG. 4 corresponds to the tube extension section 30 of FIG. 1.

As for the embodiment of FIG. 2A, the connector 340 comprises a main body 341, a first end portion 341a having a first contact surface 342, a second end portion 341b having a second contact surface 344, and first and second tube holders 346, 348 encompassing corresponding first and second contacting portions 321, 322 of the flexible tube 320 of the tube extension section 330. As the structure and functions associated with these feature in large are corresponding those described for the embodiment of FIG. 2A, the features are not described in detail again. However, as shown in FIG. 4, the first and second tube holders 346, 348 are not tubular. The first and second tube holders 346, 348 are preferably forming friction fit tube holders.

The main body 341 of the connector 340 of FIG. 4 is stretchable, and typically not breakable. The main body 341 of the connector 340 is elongated, and is typically stretchable in the direction of its elongation. Moreover, the tube extension section 330 is formed by a plurality of loops 350 of the flexible tube 120. That is, the first connecting portion 321 and the second connecting portion 322 of the flexible tube 320 is separated by at least one loop 350 of the flexible tube 320. Hereby, the flexible tube 320 may be arranged in a space saving manner.

Thus, upon being subject to a pulling force P1 larger than the threshold value as previously described, the main body 341 is configured to stretch. As the main body 341 stretches, the tube extension section 330 is reconfigured. In the example embodiment of FIG. 4, as the connector 340 is subjected to the pulling force P1 larger than the threshold value, the tube extension section 330 is re-configured as the main body 341 stretches and thereby the flexible tube 320 in the tube extension section 230 is at least partly untied, or at least partly straightened. Hereby, the reachability of the upstream tube section and/or the downstream tube section is increased as previously described.

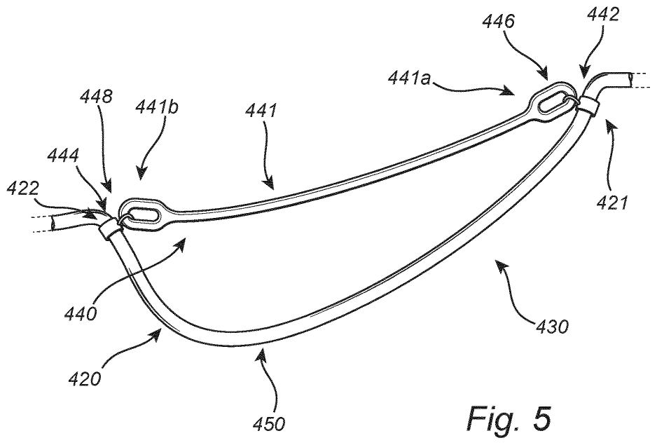

Turning to FIG. 5, showing an enlarged view of a part of a flexible tube 420 and a connector 440 in a tube extension section 430 (or a tube extension sub-section) according to at least one example embodiment. The flexible tube 420 may be that of FIG. 1, and the connector 440 may any one of the plurality of connectors 40a, 40b, 40c, 40d, 40e arranged in a corresponding tube extension sub-section. Thus, the tube extension section 430 may be any one of the tube extension sub-sections 30a, 30b, 30c, 30d, 30e of FIG. 1. As an alternative, the tube extension section 430 comprises only the one connector 440, and the tube extension section 430 of FIG. 4 corresponds to the tube extension section 30 of FIG. 1.

As for the embodiment of FIG. 2A, the connector 440 comprises a main body 441, a first end portion 441a having a first contact surface 442, a second end portion 441b having a second contact surface 444, and first and second tube holders 446, 448 encompassing corresponding first and second contacting portions 421, 422 of the flexible tube 420 of the tube extension section 430. As the structure and functions associated with these feature in large are corresponding those described for the embodiment of FIG. 2A, the features are not described in detail again. However, as shown in FIG. 5, the first and second tube holders 446, 448 are not tubular. The first and second tube holders 446, 448 are preferably forming hooks or loops.

The main body 441 of the connector 440 is stretchable, and typically not breakable. Moreover, the tube extension section 430 is formed by tube slacking section in the form of a tube bend 450. That is, the first connecting portion 421 and the second connecting portion 422 of the flexible tube 420 is separated by at least one tube slacking section, or tube bend 450. Hereby, the flexible tube 420 may be arranged in a space saving manner.

Thus, upon being subject to a pulling force P1 larger than the threshold value as previously described, the main body 441 is stretched. As the main body 441 stretches, the tube extension section 430 is reconfigured. In the example embodiment of FIG. 5, as the connector 440 is subjected to the pulling force P1 larger than the threshold value, the tube extension section 430 is re-configured as the main body 441 stretches and thereby the flexible tube 420 in the tube extension section 430 is at least partly straightened. Hereby, the reachability of the upstream tube section and/or the downstream tube section is increased as previously described.

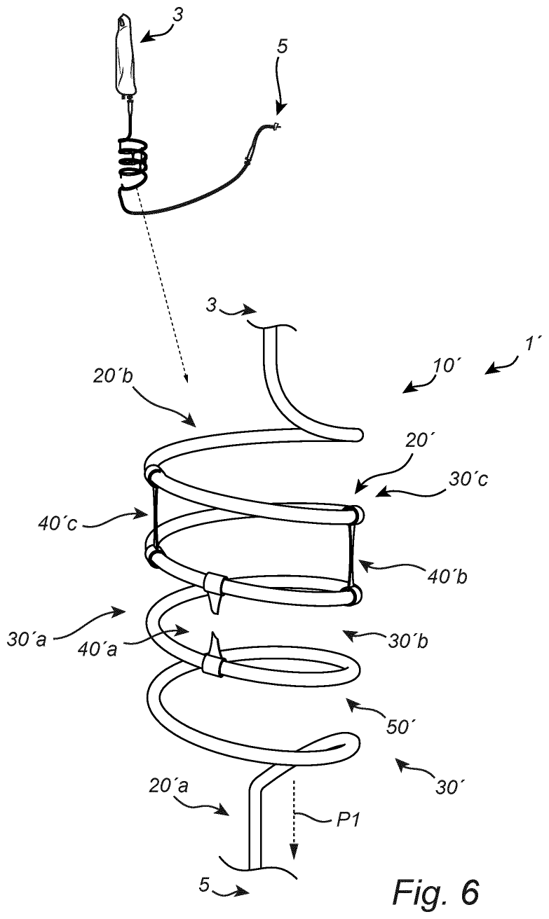

FIG. 6 shows a second example of medicament tube arrangement 10′ for an infusion set 1′, and an infusion set 1′, according to at least one example embodiment. The infusion set 1′ comprises a medicament reservoir 3 and a needle 4 for delivering medicament at an injection or infusion site 5 corresponding to the infusion set 1 of FIG. 1. The medicament tube arrangement 10′ of FIG. 6 is arranged in between the medicament reservoir 3 and the injection or infusion site 5 (or needle 4).

As for the embodiment of FIG. 1, the medicament tube arrangement 10′ comprises a flexible tube 20′ for delivering medicament and having a tube length extending between the medicament reservoir 3 and the injection or infusion site 5, wherein the flexible tube 20′ comprises a tube extension section 30′ in which the flexible tube 20 is bundled up, or folded, by a plurality of tube extension sub-sections 30′a, 30′b, 30′c using a plurality of connectors 40′a, 40′b, 40′c arranged in the tube extension section 30′. Correspondingly, the tube extension section 30′ divides the flexible tube 20′ into at least an upstream tube section 20′b extending between the medicament reservoir 3 and the tube extension section 30′, and a downstream tube section 20′a extending between the tube extension section 30′ and the infusion or injection site 5. As the structure and functions associated with these feature in large are corresponding those described for the embodiment of FIG. 1, the features are not described in detail again.

However, in the embodiment of FIG. 6, the flexible tube 20′ is formed as loops 50′ in the tube extension sub-sections 30′a, 30′b, 30′c. For example, for the intermediate tube extension sub-section 30′b, arranged between a first tube extension sub-section 30′a and a last tube extension sub-section 30′c, a loop of the flexible tube 20′ is formed. That is, with reference to the previously described first and second connecting portions of the connectors, the loop is formed from a first connecting portion of the second connector 40′b to a second connecting portion of the second connector 40′b, wherein a second connecting portion of the first connector 40′a is arranged therebetween. Hereby, the flexible tube 20′ may be arranged in a space saving manner.

As previously mentioned, and not repeated in detail here again, each one of the connectors 40′a, 40′b, 40′c is configured to, upon being subject to a pulling force P1 larger than a threshold value, re-configure the tube extension section 30 to thereby extend the reachability, such as the axial reachability, of the upstream tube section 20′b and/or the downstream tube section 20′a. Here, typically by untying, or at least partly untying, at least one of the loops of the tube extension sub-sections 30′a, 30′b, 30′c, e.g. by that the associated connector 40′a, 40′b, 40′c breaks.

It should be noted that any of the connectors 140, 240, 340, 440 described with reference to FIGS. 2A, 3, 4 and 5 may be used as any one of the connectors 40′a, 40′b, 40′c in the embodiment of FIG. 6.

For each one of the example embodiments, the dead space of the corresponding flexible tube 20, 20′, 120, 220, 320, 420 is preferably the same regardless of the configuration of the tube extension section 30, 30′, 130, 230, 330, 430. Hereby, the flexible tube 20, 20′, 120, 220, 320, 420 is configured to have a constant volume of retained medication regardless of the configuration of the tube extension section 30, 30′, 130, 230, 330, 430.

It should be noted that any of the connectors 40a, 40b, 40c, 40d, 40e, 40′a, 40′b, 40′c, 140, 240, 340, 440 described with reference to FIGS. 1-6 may be made of a polymer material. For example, at least the main body, or the previously described interconnecting portion, of the connector 40a, 40b, 40c, 40d, 40e, 40′a, 40′b, 40′c, 140, 240, 340, 440 is made of a polymer material. The chosen polymer material may be stretchable, or strain hardening, in correspondence with the earlier described embodiments. The chosen polymer material may be breakable upon being subject to a pulling force P1 larger than the threshold value, in correspondence with the earlier described embodiments.

For the embodiments of FIGS. 1 and 6, each connector 40a, 40b, 40c, 40d, 40e, 40′a, 40′b, 40′c may comprise a main body being breakable upon being subject to a pulling force larger than a threshold value, as described with reference to FIGS. 2A and 2B. The connectors 40a, 40b, 40c, 40d, 40e, 40′a, 40′b, 40′c may be arranged such that the threshold value varies for the different main bodies of the connectors 40a, 40b, 40c, 40d, 40e, 40′a, 40′b, 40′c. Thus, each connector 40a, 40b, 40c, 40d, 40e, 40′a, 40′b, 40′c in the plurality of connectors comprises main body as previously described, wherein each one of the main bodies brakes at different pulling forces (i.e. they have different breaking forces). For example, the breaking force of each contactor 40a, 40b, 40c, 40d, 40e, 40′a, 40′b, 40′c may gradually increase from the first tube extension sub-section 40a, 40′a to the last tube extension sub-section 40e, 40′c (or vice versa) so that the flexible tube 20, 20′ unwinds or unfolds from upstream to downstream (or “top to bottom”) or downstream to upstream (or “bottom to top”).

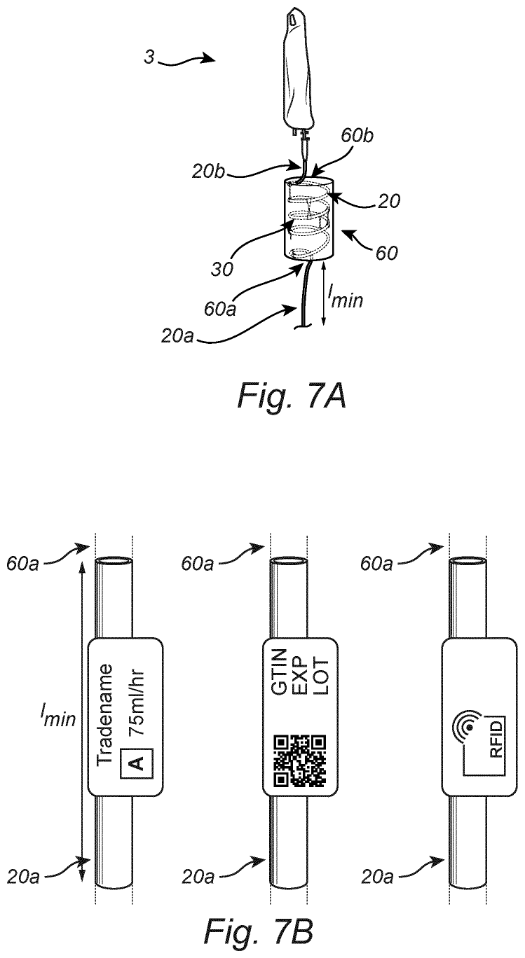

Turning to FIG. 7A showing the medicament tube arrangement 10 of FIG. 1 further comprising a housing 60 arranged to cover the tube extension section 30. Hereby, the tube extension section 30 may be efficiently stored in the housing 60. Thus, the plurality of tube extension sub-sections 30a, 30b, 30c, 30d, 30e, is arranged inside the housing 60.

As seen in FIG. 7, a the housing 60 comprises a first opening 60a through which the downstream tube section 20a extends. Hereby, a predefined length of the flexible tube 20 is available outside of the housing 60. That is, the predefined length of the flexible tube 20 is available outside of the housing downstream of the first opening 60a. The predefined length may be adapted to at least provide a sufficient gripping portion of the downstream tube section 20a, such that the patient may grip the downstream tube section 20a and pull the flexible tube 20, e.g. in a direction towards the injection or infusion site, to induce the previously mentioned pulling force P1 to the contactor larger than the threshold value. The portion of the flexible tube 20 extending through the first opening 60a of the housing 60 and having the predefined length may be provided with information of the medicament tube arrangement and/or the medicament in the medicament reservoir, for example in the form of a barcode (e.g. a QR code) or other digital readable marker, as shown in FIG. 7B.

As also seen in FIG. 7A, the housing 60 may comprise a second opening 60b arranged distant to the first opening 60a, wherein the upstream tube section 20b extends through the second opening 60a such that a predefined length of the flexible tube 20 is available outside of the housing 60 upstream of the second opening 60b.

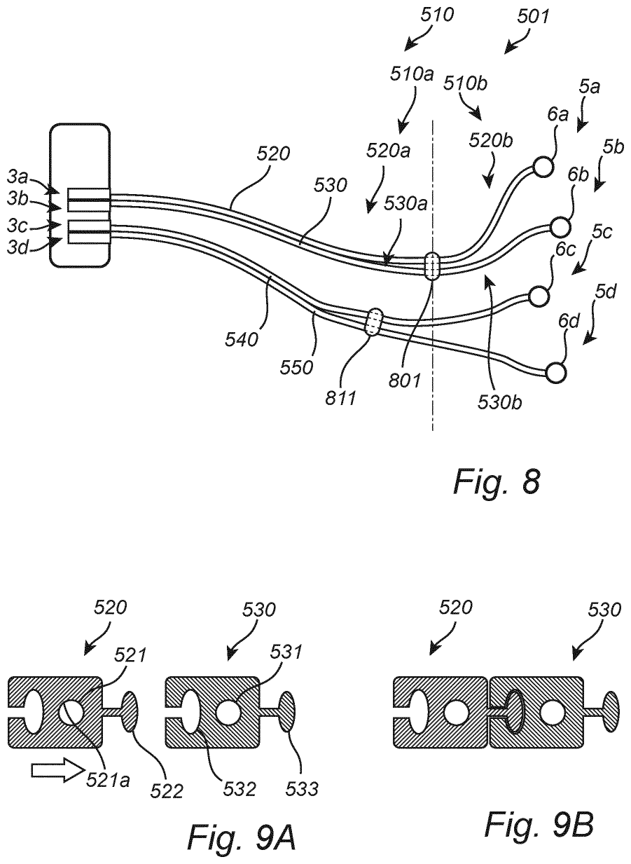

FIG. 8 shows another example of medicament tube arrangement 510, and an infusion set 501 comprising such medicament tube arrangement 510 according to an example embodiment. The infusion set 501 comprises four medicament reservoirs 3a, 3b, 3c, 3d, for delivering medicament at four corresponding injection or infusion sites 5a, 5b, 5c, 5d, e.g. provided by four corresponding injection assemblies 6a, 6b, 6c, 6d, e.g. comprising an associated needle, and the medicament tube arrangement 510 being arranged in between the medicament reservoirs 3a, 3b, 3c, 3d and the injection or infusion sites 5a, 5b, 5c, 5d. The injection assemblies 6a, 6b, 6c, 6d may be attached to the patient at the injector or infusion sites 5a, 5b, 5c, 5d by means of a pad using a needle adhesive. The medicament reservoirs 3a, 3b, 3c, 3d and the injection assemblies 6a, 6b, 6c, 6d may be comprised in the infusion set 501 or the medicament tube arrangement 510, and may together form, or form parts of, a medicament delivery device.

The medicament tube arrangement 510 comprises a first flexible tube 520 for delivering medicament from a first medicament reservoir 3a. The first flexible tube 520 has a tube length extending between the first medicament reservoir 3a and a first injection assembly 6a at a first injection or infusion site 5a, as it is arranged to fluidly connect the first medicament reservoir 3a with the first injection assembly 6a.

The medicament tube arrangement 510 further comprises additionally flexible tubes arranged in a corresponding manner as the first flexible tube 520. Thus, in more detail, the medicament tube arrangement 510 comprises: a second flexible tube 530 for delivering medicament from a second medicament reservoir 3b, a third flexible tube 540 for delivering medicament from a third medicament reservoir 3c, and a fourth flexible tube 550 for delivering medicament from a fourth medicament reservoir 3d. The second flexible tube 530 has a tube length extending between the second medicament reservoir 3b and a second injection assembly 6b at a second injection or infusion site 5b, as it is arranged to fluidly connect the second medicament reservoir 3b with the second injection assembly 6b. The third flexible tube 540 has a tube length extending between the third medicament reservoir 3c and a third injection assembly 6c at a third injection or infusion site 5c, as it is arranged to fluidly connect the third medicament reservoir 3c with the third injection assembly 6c. The fourth flexible tube 550 has a tube length extending between the fourth medicament reservoir 3d and a fourth injection assembly 6d at a fourth injection or infusion site 5d, as it is arranged to fluidly connect the fourth medicament reservoir 3d with the fourth injection assembly 6d.

Thus, during use, medicament can be delivered from the medicament reservoirs 3a, 3b, 3c, 3d to the injection assemblies 6a, 6b, 6c, 6d at the injection or infusion sites 5a, 5b, 5c, 5d by being fluidly transported in the first, second, third and fourth flexible tubes 520, 530, 540, 550 of the medicament tube arrangement 510. Each one of the medicament reservoirs 3a, 3b, 3c, 3d may e.g. be equipped with a pressure drive, such as e.g. a pump, for providing the necessary pressurization of the medicament.

Turning to FIG. 9A, showing a radial cross section of the first flexible tube 520 and the second flexible tube 530 of the medicament tube arrangement 510 of FIG. 8. The first flexible tube 520 has a first tube lumen 521 configured to transport the medicament from the first reservoir 3a. Correspondingly, the second flexible tube 530 has a second tube lumen 531 configured to transport the medicament from the second reservoir 3b. As shown in FIGS. 1 and 2, the first tube lumen 521 is separated from the second tube lumen 531. As shown for the first tube lumen 521, but applicable to any of the tube lumens of the described flexible tubes, the first flexible tube comprises inner walls 521a defining the first tube lumen 521. Thus, the first lumen inner walls 521 is arranged to be in direct contact with the medicament delivered from the first medicament reservoir 3a. The inner walls 521a completely encloses the first tube lumen 521.

The first flexible tube 520 comprises a first tube locking protrusion 522 forming a connector. As seen in FIG. 9A, the first tube locking protrusion 522 forms a radial protrusion of the first flexible tube 520. The second flexible tube 530 comprises a second tube receiving recess 532. As seen in FIG. 9A, the second tube receiving recess 531 forms a radial recess of the second flexible tube 530. The radial cross section of the first tube locking protrusions 522 is formed as a positive puzzle piece and the radial cross section of the second tube receiving recess 532 is formed as a corresponding negative puzzle piece. Stated differently, the first tube locking protrusion 522 comprises a knob, or bulb, arranged at the end portion of the first tube locking protrusion 522. Correspondingly, the second tube receiving recess 533 is sized and dimensioned to hold the knob, or bulb, of the first tube locking protrusion 522. Typically, the second flexible tube 530 comprises at least two flexible arms, or flanges, defining the entrance into the second tube receiving recess 533, the entrance having a cross section smaller than that of the knob, or bulb. Thus, as the first tube locking protrusion 522 is releasably locked into the second tube receiving recess 533, the knob, or bulb, is moved by the entrance and into the second tube receiving recess 533 by pushing the two flexible arms, or flanges, apart. Hereby, the first tube locking protrusion 522 can be releasably locked into the second tube receiving recess 533.

Thus, the connector, or first tube locking protrusion 522, and the second tube receiving recess 531 are configured to releasably lock to each other, as shown in FIG. 9B. Hereby, the first flexible tube 520 is connectable, such as releasably lockable connectable, to the second flexible tube 530. Thus, the first flexible tube 520 may be co-routed with the second flexible tube 530, as least along a first portion 510a of the medicament tube arrangement 510, as shown in FIG. 8. Hereby, the radial reachability of the first and second flexible tubes 520, 530 is increased.

In more detail, along the first portion 510 of the medicament tube arrangement 510, the first tube locking protrusion 522 is locked to the second tube receiving recess 532 such that a first portion 520a of the first flexible tube 520 is co-routed with a corresponding first portion 530a of the second flexible tube 530. Moreover, along a second portion 510b of the medicament tube arrangement 510, the first tube locking protrusion 522 is distant from the second tube receiving recess 532 such that a second portion 520b of the first flexible tube 520 is freely arrangeable in relation to a corresponding second portion 530b the second flexible tube 530. Hereby, the first flexible tube 520 and the second flexible tube 530 may more easily reach different infusion sites, such as the first injection or infusion site 5a and the second injection of infusion site 5b as shown in FIG. 8.

In other words, the connector, or first tube locking protrusion 522, is arrangeable in a first configuration in which the first portion 520a of the first flexible tube 520 is arranged adjacent the first portion 530b of the second flexible tube 530, and is arrangeable in a second configuration in which the second portion 520b of the first flexible tube 520 is arranged spaced apart from the second portion 530b of the second flexible tube 530. Thus, at least a part of the connector, or first tube locking protrusion 522, is configured to be in contact with the first flexible tube 520 and the second flexible tube 530 in both the first and second configurations.

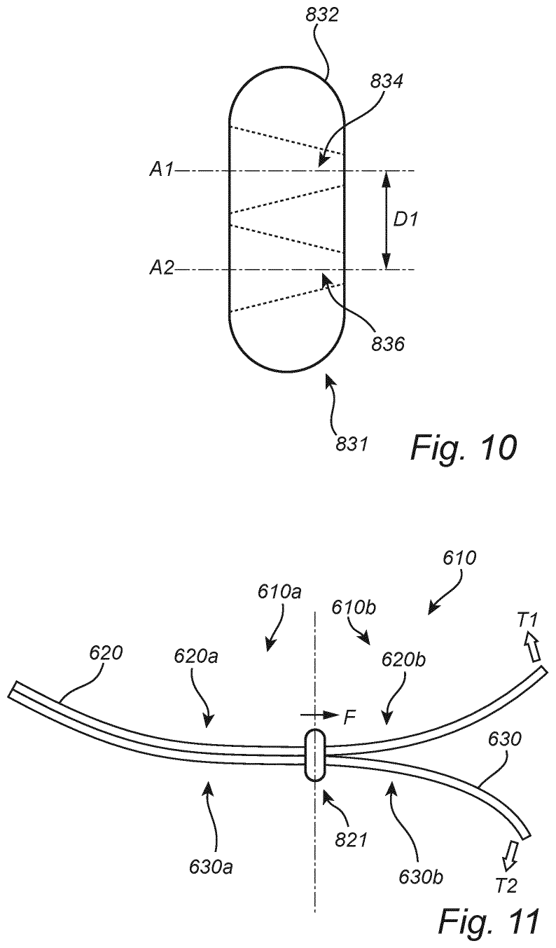

The medicament tube arrangement 510 may comprise a lock-and-release unit 801 configured to change the configuration of the connector, or first tube locking protrusion 522, from its first configuration to its second configuration, or vice versa. An example embodiment of such lock-and-release unit 831 is shown in a cross sectional side view in FIG. 10.

The lock-and-release unit 831 comprises a main body 832 which his movable along the first and second flexible tubes 520, 530 in a forward direction and a backward direction. The forward and backwards directions are along the length, or centre axis, of the first and second flexible tubes 520, 530. The main body 832 comprises a first through-hole 834 through which the first flexible tube 520 extends and a second through-hole 836 through which the second flexible tube 530 extends. As shown in FIG. 10, each of the first and second through-holes 834, 836 extend along a corresponding geometric axis, A1, A2, and the first and second through-holes 834, 836 are separated by a spacing D1. The spacing D1, as well as the first and second through-holes 834, 836, are sized and dimensioned such that the first tube locking protrusion 522 of the first flexible tube 520 locks to the second tube receiving recess 532 of the second flexible tube 530, 630 when the main body 832 is moved in the forward direction. Correspondingly, the spacing D1, as well as the first and second through-holes 834, 836, are sized and dimensioned such that the first tube locking protrusion 522 of the first flexible tube 520 is released from the second tube receiving recess 532 of the second flexible tube 530 when the main body 832 is moved in the backward direction. The first and second through-holes 834, 836 may e.g. be tapering along the corresponding geometric axis, A1, A2.

FIG. 11 shows an example of medicament tube arrangement 610 for an infusion set, such as the infusion set 501 of FIG. 8, comprising only a first flexible tube 620, a second flexible tube 630, and an optional lock-and-release unit 821. The first and second flexible tubes 620, 630 may be the same as, or correspond to, the first and second flexible tubes 520, 530 described in the embodiments of FIGS. 8 and 9A-9B, and are thus not described here again. Thus, the four flexible tubes 520, 530, 540, 550 of the medicament tube arrangement 510 of FIG. 8 may be exchanged with the medicament tube arrangement 610 and the first and second flexible tubes 620, 630. Moreover, the optional lock-and-release unit 821 may be the same as, or correspond to, the lock-and-release unit 831 of FIG. 10.

As shown in FIG. 11, as the lock-and-release unit 821 is moved in the forward direction F, along a first portion 610a of the medicament tube arrangement 610, the first tube locking protrusion of the first flexible tube 620 is locked to the second tube receiving recess of the second flexible tube 630 such that a first portion 620a of the first flexible tube 620 is co-routed with a corresponding first portion 630a of the second flexible tube 630. Thus, in the remaining portion of the medicament tube arrangement 610, or along a second portion 610b of the medicament tube arrangement 610, which the lock-and-release unit 821 has not (yet) reached, the first tube locking protrusion of the first flexible tube 620 is distant from the second tube receiving recess of the second flexible tube 630 such that a second portion 620b of the first flexible tube 620 is freely arrangeable, indicated by arrow T1, in relation to a corresponding second portion 630b the second flexible tube 630, indicated by arrow T2. Hereby, the first flexible tube 620 and the second flexible tube 630 may more easily reach different infusion sites.

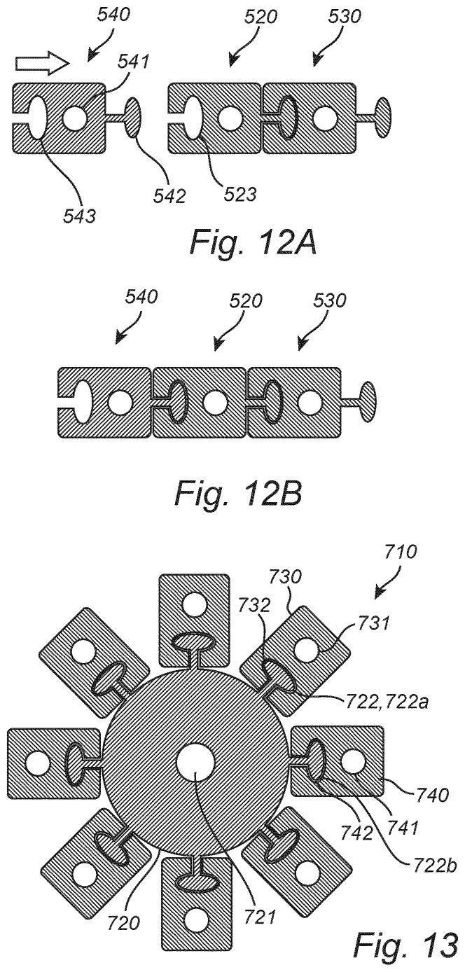

Turning back to FIG. 8, and with further reference to FIGS. 12A and 12B, the third flexible tube 540 comprises a third tube lumen 541 configured transport the medicament from the third medicament reservoir 3c, and a third tube locking protrusion 542 and a third tube receiving recess 543. Hereby, the third flexible tube 540 may interact with the first flexible tube 520 as shown in FIG. 12A in a corresponding manner as the previously described interaction between the first and second flexible tubes 520, 530. In more detail, the first flexible tube 520 may comprises a first tube receiving recess 523 configured to releasably lock to the third tube locking protrusion 542 of the third flexible tube 540. Thus, as shown in FIG. 12B, the first, second and third flexible tubes 520, 530, 540 may be arranged in a stack in which the first flexible tube 520 is arranged in between the second flexible tube 530 and the third flexible tube 540.

Correspondingly, even though not disclosed in the drawings, the fourth flexible tube 550 typically comprises a fourth tube lumen configured transport the medicament from the fourth medicament reservoir 3d, and a fourth tube locking protrusion and/or a fourth tube receiving recess. Hereby, the fourth flexible tube 550 may be releasably locked to the third flexible tube 540 by interaction with the third tube receiving recess 543, or to the second flexible tube 530 by interaction with a second tube locking protrusion 533. Thus, the first, second, third and fourth flexible tubes 520, 530, 540, 550 may be arranged in a stack. For an embodiment with three or four flexible tubes arranged in a stack, the lock-and-release unit should be adapted accordingly, i.e. with an additional through-hole for each additional flexible tube. However, as shown in FIG. 8, a secondary lock-and-release unit 811 may be configured to releasably lock the third and fourth flexible tubes 540, 550 in a corresponding manner as the previously described lock-and-release unit 801, 831 for the first and second flexible tubes 520, 530. Thus, the secondary lock-and-release unit 811 may be the same as, or correspond to, the lock-and-release unit 831 of FIG. 10. Hereby, two sets of releasably lockable flexible tubes may be provided, a first set comprising the first and second flexible tubes 520, 530 and the previously described lock-and-release unit 801, 831, and a second set comprising the third and fourth flexible tubes 540, 550 and the secondary lock-and-release unit 811. Hereby, the first set may be routed to a first location of the user, e.g. an upper body of the user, and the second set may be routed to a second location of the user, e.g. an abdomen or a lower body of the user.

FIG. 13 shows a cross sectional view of another example of a medicament tube arrangement 710. In this embodiment, a first flexible tube 720 comprises a first tube lumen 721 and a plurality of first tube locking protrusions 722, of which a first and second first tube locking protrusions 722a, 722b are indicated.

Moreover, the medicament tube arrangement 710 comprises a plurality of flexible tubes arranged radially of the first flexible tube 720, of which a second and third flexible tubes 730, 740 are indicated. The second and third flexible tubes 730, 740 are arranged around the periphery of the first, central, flexible tube 720 such that the second and third flexible tubes 730, 740 are arranged radially of the first flexible tube 720, but at different directions. Thus, the second and third flexible tubes 730, 740 are separated by an angle greater than zero.

The second flexible tube 730 comprises a second tube lumen 731 and a second tube receiving recess 732 and the third flexible tube 740 comprises third tube lumen 741 and a fourth tube receiving recess 742. The first and second tube locking protrusions 722a, 722b are releasably locked to the second tube receiving recess 732 and the third tube receiving recess 742, respectively, in a corresponding manner as previously described. Hereby, a centrally arranged flexible tube 720 which is releasably connected to at least second and third flexible tubes 730, 740 are provided. As shown in FIG. 13, more than two flexible tubes may be correspondingly connected to the first flexible tube 720.

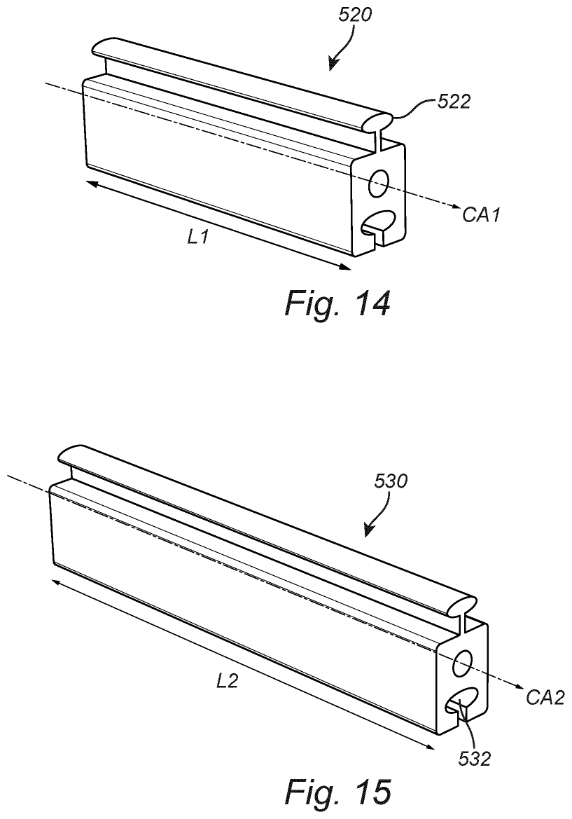

The first flexible tube 520 of the embodiment of FIG. 8 is shown in perspective view in FIG. 14. Here, a centre axis CA1 extends along the length L1 of the first flexible tube 520. The tube length L1 is arranged to extend from the first medicament reservoir (shown in FIG. 8) to the first injection or infusion site or corresponding injection assembly (shown in FIG. 8). As seen in the embodiment of FIG. 14, the first tube locking protrusion 522 extends along at least a majority of the tube length L1, here the entire tube length L1. Thus, the first flexible tube 520 may be releasably locked to e.g. the second flexible tube 530 along its entire tube length L1.

The second flexible tube 530 of the embodiment of FIG. 8 is shown in a perspective view in FIG. 15. Here, a centre axis CA2 extends along the length L2 of the second flexible tube 530. The tube length L2 is arranged to extend from the second medicament reservoir (shown in FIG. 8) to the second injection or infusion site or corresponding injection assembly (shown in FIG. 8). As seen in the embodiment of FIG. 15, the second tube receiving recess 532 extends along at least a majority of the tube length L2, here the entire tube length L2. Thus, the second flexible tube 530 may be releasably locked to e.g. the first flexible tube 520 along its entire tube length L2.

As obvious when comparing FIGS. 14 and 15, the tube length L1 of the first flexible tube 520 may be different to the tube length L2 of the second flexible tube 530. For example, the tube length L2 of the second flexible tube 530 is larger than the tube length L1 of the first flexible tube 520. Hereby, the first flexible tube 520 and the second flexible tube 530 may be arranged to reach injection or infusion sites 5a, 5b at different locations of the user.

As also shown in FIGS. 14 and 15, the radial cross section anywhere along the first flexible tube 520 or along the second flexible tube 530 is non-circular. The radial cross section of the first and second flexible tubes 520, 530 are rectangular or square-like with the addition of the tube locking protrusions and tube receiving recesses.

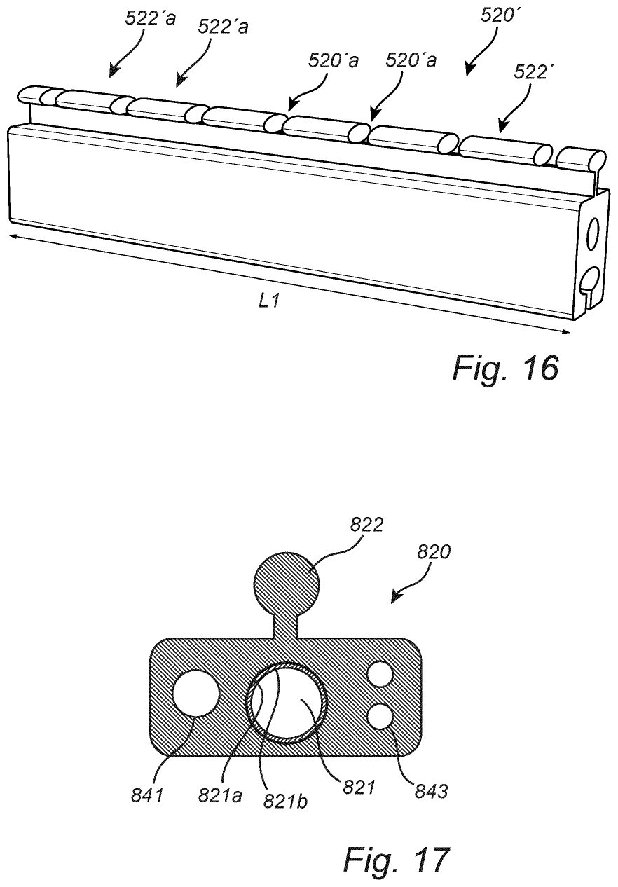

Turning to FIG. 16 showing an alternative embodiment of a first flexible tube 520′. Thus, the first flexible tube 520′ may replace the first flexible tube 520 in the previous mentioned embodiments. The first flexible tube 520′ of the embodiment of FIG. 16 is shown in a perspective view. The first flexible tube 520′ has a corresponding tube length L1 as the first flexible tube 520 as previously described. However, a first tube locking protrusion 522′ of the first flexible tube 520′ in FIG. 16 is divided into sub-portions 522′a arranged intermittingly along the tube length L1 of the first flexible tube 520′. Thus, the intermittent sub-portions 522′a of the first tube locking protrusion 522′ may be separated by tube portions 520′a having no first tube locking protrusion 522′. Hereby, the first flexible tube 520′ may efficiently be released from the second flexible tube 530 at the tube portions 520′a having no first tube locking protrusion 522′, i.e. in the spacing between the sub-portions 522′a of the first tube locking protrusion 522′. Even though not disclosed in the drawings, the second tube receiving recess 532 of the second flexible tube 530 may according to one embodiment be arranged in a corresponding manner with sub-portions arranged intermittingly along the tube length L2 of the second flexible tube 530, regardless of if the first flexible tube 520, 520′ comprises such sub-portions 522′a arranged intermittingly along the tube length L1 or not.

According to one embodiment, any one of the flexible tubes, such as e.g. the first flexible tube 520 and the second flexible tube 530, are extruded. That is, the flexible tubes may be extruded flexible tubes, i.e. flexible tubes manufactured by extrusion.

FIG. 17 shows a radial cross section of an alternative embodiment of a first flexible tube 820 which typically has been extruded. The first flexible tube 820 of FIG. 17 may replace the first flexible tube 520 in the previous mentioned embodiments. The first flexible tube 820 has a corresponding first tube lumen 821 defined by inner walls 821a and a corresponding first tube locking protrusion 822. In the embodiment of FIG. 17, the first flexible tube 820 comprises no first tube receiving recess. However, it should be noted that the first flexible tube 820 may be configured with the first tube lumen 821 arranged centrally of the first flexible tube 820, and in which a first tube receiving recess is included as for the previously described first flexible tubes 520, 620.

In the embodiment of FIG. 17, the inner walls 821a of the first tube lumen 821 is coated with a polymer material 821b. Hereby, the material intended to be in contact with the medicament from the first medicament reservoir 3a, i.e. the polymer material 821b, may be adapted accordingly. For example, the first flexible tube 820 with the polymer material 821b of the first tube lumen 821 may be manufactured by co-extrusion. That is, the remaining parts of the first flexible tube 820 may be co-extruded with the polymer material 821b of the inner walls 821a of the first tube lumen 821. Thus, an efficient means for manufacturing the first flexible tube 820 with the polymer material 821b of the first tube lumen 821 is achieved.

The polymer material 821b may be chosen to be a material selected for low leachable and extractable compounds that may contaminate the medicament, and that exhibits high biocompatibility with biologic medications. For example, the polymer material 821b may be a silicone, COC, COP, PVC, PVC without DEHP, EVA, HDPE, LDPE, TPU, PTFE, PCTFE or a fluoropolymer. The polymer material 821b may be the same as the material of the remaining parts of the first flexible tube 820. The material of the remaining parts of the first flexible tube 820 may be a silicone, COC, COP, PVC, PVC without DEHP, EVA, HDPE, LDPE, TPU, PTFE, PCTFE or a fluoropolymer.

As shown in FIG. 17, the first flexible tube 820 may comprise additional secondary lumens 841, 843 for pneumatic, fluid, electrical and/or optical means. For example, a secondary medicament may be delivered in a secondary first tube lumen 841, and an electrical cable may be routed in an electrical cable lumen 843.

It should be understood that the first flexible tube 520, 620, 720, 820 may be referred to as a first flexible tube structure, and that the second flexible tube 530, 630, 730 may be referred to as a second flexible tube structure. In other words, the connector, or first tube locking protrusion 522, is arrangeable in a first configuration in which the first flexible tube structure, or at least a portion thereof, is arranged adjacent the second flexible tube structure, and is arrangeable in a second configuration in which the first flexible tube structure, or at least another portion thereof, is arranged spaced apart from the second flexible tube structure.

Turning briefly back to FIG. 1, instead of the embodiment described e.g. with reference to FIG. 8 in which the first flexible tube structure is comprised in, or composed of, the first flexible tube 520 and the second flexible tube structure is comprised in, or composed of, the second flexible tube 530, the first and second flexible tube structure is in the embodiment of FIG. 1 comprised in a single, or common, flexible tube 20. Thus, any one of the connectors 40a, 40b, 40c, 40d, 40e is arrangeable in a first configuration in which the first flexible tube structure, or first connecting portion, is arranged adjacent to the second flexible tube structure, or second connecting portion, i.e. a first portion of the flexible tube 20 is arranged adjacent to a second portion of the flexible tube 20 in order to connect two axially separated portions of the flexible tube 20. Correspondingly, in the second configuration of the connector, the first flexible tube structure, or first connecting portion, is arranged spaced apart from the second flexible tube structure, or second connecting portion, i.e. the first portion of the flexible tube 20 is no longer arranged adjacent to the second portion of the flexible tube 20 so that the two axially separated portions of the flexible tube 20 is no longer connected by the connector, or is at least further spaced apart by the connector as compared to when the connector is arranged in its first configuration.

The medicament tube arrangement, or the infusion set, described herein may form, or be comprised in, a wearable medicament delivery device. Hereby, the medicament tube arrangement can be worn near-body the patient, over or under the clothing of the patient, without being directly attached to the patient's body, e.g. without being directly attached to the patient's skin.

The medicament tube arrangement, or the infusion set, may take on numerous different outer shapes, depending on the design, number of needles, desired injection rotation schedule between injections, the volume of medicament to be infused at a given site through a given needle, nature of the medicines themselves, medication regimen, medication administration sequence(s), a desire to prevent mixing of medications at their respective injection sites, or based on results from pharmacokinetic/pharmacodynamic modelling or human clinical trial(s) of one or more medicaments.

The medicament tube arrangement, or the infusion set, described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjögren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the medicament reservoir, and administrated by the medicament tube arrangement in the described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the medicament reservoir, and administrated by the medicament tube arrangement described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the medicament reservoir, and administrated by the medicament tube arrangement described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C—C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation w137 (CDw137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the medicament reservoir, and administrated by the medicament tube arrangement described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the medicament reservoir, and administrated by the medicament tube arrangement described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the medicament reservoir, and administrated by the medicament tube arrangement described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the medicament reservoir, and administrated by the medicament tube arrangement described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the medicament reservoir, and administrated by the medicament tube arrangement described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the medicament reservoir, and administrated by the medicament tube arrangement described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, human-derived, or recombinant hyaluronidase enzyme), concentration modifiers or enhancers, stabilizers, buffers, or other excipients.

Exemplary drugs that could be included in the medicament reservoir, and administrated by the medicament tube arrangement described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOpAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

The inventive concept has mainly been described above with reference to a few examples. However, as is readily appreciated by a person skilled in the art, other embodiments than the ones disclosed above are equally possible within the scope of the inventive concept, as defined by the appended claims.

Some other aspects of the inventions are defined in the following clauses.
1. A medicament tube arrangement (10, 10′, 510, 610, 710) for an infusion set (1, 1′, 501) comprising:
a first flexible tube structure (121, 221, 321, 421, 520, 520′, 620, 720, 820) and a second flexible tube structure (122, 222, 322, 422, 530, 630, 730) for delivering medicament from a medicament reservoir (3, 3a, 3b),
at least one connector (40a, 40b, 40c, 40d, 40e, 40′a, 40′b, 40′c, 140, 240, 340, 440, 522, 522′, 722, 822) being configured to connect the first flexible tube structure (121, 221, 321, 421, 520, 520′, 620, 720, 820) to the second flexible tube structure (122, 222, 322, 422, 530, 630, 730),
wherein the connector is arrangeable in a first configuration in which the first flexible tube structure (121, 221, 321, 421, 520, 520′, 620, 720, 820) is arranged adjacent the second flexible tube structure (122, 222, 322, 422, 530, 630, 730), and is arrangeable in a second configuration in which the first flexible tube structure (121, 221, 321, 421, 520, 520′, 620, 720, 820) is arranged spaced apart from the second flexible tube structure (122, 222, 322, 422, 530, 630, 730).
2. The medicament tube arrangement (510, 610, 710) according to clause 1, wherein the connector (522, 522′, 722, 822) is configured to extend the radial reachability of medicament tube arrangement.
3. The medicament tube arrangement (10, 10′, 510, 610, 710) according to any one of the preceding clauses, wherein at least a part of the connector (40a, 40b, 40c, 40d, 40e, 40′a, 40′b, 40′c, 140, 240, 340, 440, 522, 522′, 722, 822) is configured to be in contact with the first flexible tube structure (121, 221, 321, 421, 520, 520′, 620, 720, 820) and the second flexible tube structure (122, 222, 322, 422, 530, 630, 730) in both the first and second configurations.
4. The medicament tube arrangement (10, 10′) according to clause 1, wherein the connector (40a, 40b, 40c, 40d, 40e, 40′a, 40′b, 40′c, 140, 240, 340, 440) is configured to extend the axial reachability of medicament tube arrangement.
5. The medicament tube arrangement (510, 610, 710) according to any one of clauses 1-3 further comprising:
a first flexible tube (520, 520′, 620, 720, 820) forming the first flexible tube structure (520, 520′, 620, 720, 820) for delivering medicament from a first medicament reservoir (3a), the first flexible tube (520, 520′, 620, 720, 820) having a first tube lumen (521, 721, 821) configured to transport the medicament from the first reservoir (3a),
a first tube locking protrusion (522, 522′, 722, 822) forming the connector (522, 522′, 722, 822), the first tube locking protrusion (522, 522′, 722, 822) forming a radial protrusion of the first flexible tube (520, 520′, 620, 720, 820),
a second flexible tube (530, 630, 730) forming the second flexible tube structure (530, 630, 730) for delivering medicament from a second medicament reservoir (3b), the second flexible tube (530, 630, 730) having a second tube lumen (531, 731) configured to transport the medicament from the second reservoir (3b), and a second tube receiving recess (532, 732) configured to releasably lock to the first tube locking protrusion (522, 522′, 722, 822) to co-route the first flexible tube (520, 520′, 620, 720, 820) with the second flexible tube (530, 630, 730).
6. The medicament tube arrangement (510, 610, 710) according to clause 5, wherein the first flexible tube (520, 520′) has a tube length (L1) arranged to extend from the first medicament reservoir (3a) to a first injection or infusion site (5a), and wherein the first tube locking protrusion (522, 522′) extends along at least a majority of the tube length (L1) of the first flexible tube (520, 520′).
7. The medicament tube arrangement (510, 610, 710) according to any one of clauses 5-6, wherein the second flexible tube (530) has a tube length (L2) arranged to extend from the second medicament reservoir (3b) to a second injection or infusion site (5b), and wherein the second tube receiving recess (532) extends along at least a majority of the tube length (L2) of the second flexible tube (530).
8. The medicament tube arrangement (510, 610, 710) according to clauses 6-7, wherein the tube length (L1) of the first flexible tube (520, 520′) is different to the tube length (L2) of the second flexible tube (530).
9. The medicament tube arrangement (510, 610, 710) according to any one of clauses 6 and 8, wherein the first tube locking protrusion (522′) is divided into sub-portions arranged intermittingly along the tube length (L1) of the first flexible tube (520′).
10. The medicament tube arrangement (510, 610, 710) according to any one of clauses 5-9, wherein along a first portion (510a, 610a) of the medicament tube arrangement (510, 610), the first tube locking protrusion (522) is locked to the second tube receiving recess (532) such that a first portion (520a, 620a) of the first flexible tube (520, 620) is co-routed with a corresponding first portion (530a, 630a) of the second flexible tube (530, 630).
11. The medicament tube arrangement (510, 610, 710) according to clause 10, wherein along a second portion (510b, 610b) of the medicament tube arrangement (510, 610), the first tube locking protrusion (522) is distant from the second tube receiving recess (532) such that a second portion (520b, 620b) of the first flexible tube (520, 620) is freely arrangeable in relation to a corresponding second portion (530b, 630b) the second flexible tube (530, 630).
12. The medicament tube arrangement (510, 610, 710) according to any one of clauses 10-11, further comprising a lock-and-release unit (801, 811, 831) having a main body (832) movable along the first and second flexible tubes (520, 520′, 620, 820, 530, 630) in a forward direction and a backward direction, the main body (832) having a first through-hole (834) through which the first flexible tube (520, 520′, 620, 820) extends and a second through-hole (836) through which the second flexible tube (530, 630) extends, wherein the first and second through-holes (834, 836) are separated by a spacing (D1), and wherein the spacing (D1) is sized such that the first tube locking protrusion (522, 522′, 822) of the first flexible tube (520, 520′, 620, 820) locks to the second tube receiving recess (532) of the second flexible tube (530, 630) when the main body (832) is moved in the forward direction, and such that the first tube locking protrusion (522, 522′, 822) of the first flexible tube (520, 520′, 620, 820) is released from the second tube receiving recess (532) of the second flexible tube (530, 630) when the main body (832) is moved in the backward direction.
13. The medicament tube arrangement (510, 610, 710) according to any one of clauses 5-12, further comprising a third flexible tube (540, 740) for delivering medicament from a third medicament reservoir (3c), the third flexible tube (540, 740) having a third tube lumen (541, 741) configured transport the medicament from the third reservoir (3c), and a third tube locking protrusion (542) and/or a third tube receiving recess (543, 742).
14. The medicament tube arrangement (510, 610, 710) according to clause 13, wherein the first tube locking protrusion (722) is a first locking protrusion (722a) and the first flexible tube (720) comprises a second locking protrusion (722b) configured to releasably lock to the third tube receiving recess (742).
15. The medicament tube arrangement (510, 610, 710) according to clause 14, wherein the first flexible tube (520) comprises a first tube receiving recess (523) configured to releasably lock to the third tube locking protrusion (543) of the third flexible tube (540).
16. The medicament tube arrangement (510, 610, 710) according to any one of clauses 5-15, wherein a radial cross section anywhere along the first flexible tube (520, 620, 720, 820) is non-circular.
17. The medicament tube arrangement (510, 610, 710) according to any one of clauses 5-16, wherein the first flexible tube (520, 520′, 620, 720, 820) and the second flexible tube (530, 630, 730) are extruded.
18. The medicament tube arrangement (510, 610, 710) according to any one of clauses 5-17, wherein inner walls (821a) of the first tube lumen (821) is coated with a polymer material.
19. The medicament tube arrangement (510, 610, 710) according to clauses 17-18, wherein the first flexible tube (820) with the polymer material of the first tube lumen (821) has been coextruded with the remaining parts of the first flexible tube (820).
20. The medicament tube arrangement (510, 610, 710) according to any one of clauses 5-19, wherein the first lumen (521, 821) is defined by first lumen inner walls (521a, 821a), and wherein the first lumen inner walls (521a, 821a) is arranged to be in direct contact with the medicament delivered from the first medicament reservoir (3a).
21. A medicament tube arrangement (10, 10′) for an infusion set (1, 1′) according to any one of clauses 1 and 4 comprising:
a flexible tube (20, 20′, 120, 220, 320, 420) for delivering medicament, the flexible tube (20, 20′, 120, 220, 320, 420) comprising the first flexible tube structure (121, 221, 321, 421) and the second flexible tube structure (122, 222, 322, 422), the flexible tube (20, 20′, 120, 220, 320, 420) having a tube length arranged to extend between the medicament reservoir (3) and an injection or infusion site (5), and a tube extension section (30, 30′, 130, 230, 330, 430) dividing the flexible tube (20, 20′, 120, 220, 320, 420) into at least an upstream tube section (20b, 20′b) arranged to extend between the medicament reservoir (3) and the tube extension section (30, 30′, 130, 230, 330, 430), and a downstream tube section (20a, 20′a) arranged to extend between the tube extension section (30, 30′, 130, 230, 330, 430) and the infusion or injection site (5),
wherein the connector (40a, 40b, 40c, 40d, 40e, 40′a, 40′b, 40′c, 140, 240, 340, 440) is arranged in the tube extension section (30, 30′, 130, 230, 330, 430), the connector (40a, 40b, 40c, 40d, 40e, 40′a, 40′b, 40′c, 140, 240, 340, 440) being configured to, upon being subject to a pulling force (P1) larger than a threshold value, re-configure the tube extension section (30, 30′, 130, 230, 330, 430) and thereby extend the reachability of the upstream tube section (20b, 20′b) and/or the downstream tube section (20a, 20′a).
22. The medicament tube arrangement (10, 10′) according to clause 21, wherein the connector (40a, 40b, 40c, 40d, 40e, 40′a, 40′b, 40′c, 140, 240, 340, 440) has a main body (141, 241, 341, 441) comprising a first end portion (141a, 241a, 341a, 441a) having a first contact surface (142, 242, 342, 442), and a second end portion (141b, 241b, 341b, 441b) arranged opposite to the first end portion (141a, 241a, 341a, 441a), the second end portion (141b, 241b, 341b, 441b) having a second contact surface (144, 244, 344, 444), wherein the first contact surface (142, 242, 342, 442) is attached to a first connecting portion (121, 221, 321, 421) of the flexible tube (20, 20′, 120, 220, 320, 420) in the tube extension section (30, 30′, 130, 230, 330, 430), the first connecting portion (121, 221, 321, 421) forming the first flexible tube structure (121, 221, 321, 421), and the second contact surface (144, 244, 344, 444) is attached to a second connecting portion (122, 222, 322, 422) of the flexible tube (20, 20′, 120, 220, 320, 420) in the tube extension section (30, 30′, 130, 230, 330, 430), the second connecting portion (122, 222, 322, 422) forming the second flexible tube structure (122, 222, 322, 422), wherein the first and second contacting portions (121, 221, 321, 421, 122, 222, 322, 422) of the flexible tube (20, 20′, 120, 220, 320, 420) in the tube extension section (30, 30′, 130, 230, 330, 430) are distant from each other.
23. The medicament tube arrangement (10, 10′) according to clause 22, wherein the tube extension section (130, 230, 330, 430) is defined to extend from the first connecting portion (121, 221, 321, 421) to the second connecting portion (122, 222, 322, 422).
24. The medicament tube arrangement (10, 10′) according to any one of clauses 22-23, wherein the connector (40a, 40b, 40c, 40d, 40e, 40′a, 40′b, 40′c, 140, 240) comprises a first tubular tube holder (146, 246) encompassing the first contacting portion (121, 221) of the flexible tube (120, 220), the first tubular tube holder (146, 246) comprising the first contact surface (142, 242), and a second tubular tube holder (148, 248) encompassing the second contacting portion (122, 222) of the flexible tube (120, 220), the second tubular tube holder (148, 248) comprising the second contact surface (144, 244).
25. The medicament tube arrangement (10, 10′) according to any one of clauses 22-24, wherein the first connecting portion (121, 221, 321, 421) and the second connecting portion (122, 222, 322, 422) are separated by a loop (50′, 350) of the flexible tube (20′, 320), or a tube bend (150, 450) of the flexible tube (20, 120, 420).
26. The medicament tube arrangement (10, 10′) according to any one of clauses 22-25, wherein the main body (141, 241, 341, 441) of the connector (40a, 40b, 40c, 40d, 40e, 40′a, 40′b, 40′c, 140, 240, 340, 440) is stretchable.
27. The medicament tube arrangement (10, 10′) according to clause 26, wherein the main body (241) is strain hardening such that the main body (241) is extended progressively.
28. The medicament tube arrangement (10, 10′) according to any one of clauses 22-27, wherein the main body (141) of the connector (40a, 40b, 40c, 40d, 40e, 40′a, 40′b, 40′c, 140) is breakable.
29. The medicament tube arrangement (10, 10′) according to clause 28, wherein the main body (141) is breakable upon being subject to a pulling force (P1) larger than the threshold value.
30. The medicament tube arrangement (10, 10′) according to any one of clauses 22-29, wherein the main body (141, 241, 341, 441) of the connector (40a, 40b, 40c, 40d, 40e, 40′a, 40′b, 40′c, 140, 240, 340, 440) is elongated.
31. The medicament tube arrangement (10, 10′) according to any one of clauses 22-30, wherein the dead space of the flexible tube (20, 20′, 120, 220, 320, 420) is the same regardless of the configuration of the tube extension section (30, 30′, 130, 230, 330, 430).
32. The medicament tube arrangement (10, 10′) according to any one of clauses 22-31, wherein the connector (40a, 40b, 40c, 40d, 40e, 40′a, 40′b, 40′c, 140, 240, 340, 440) is made of a polymer material.
33. The medicament tube arrangement (10, 10′) according to any one of clauses 22-32, wherein the connector (40a, 40b, 40c, 40d, 40e, 40′a, 40′b, 40′c, 140, 240, 340, 440) is made of a polymer material providing elastic hysteresis.
34. The medicament tube arrangement (10, 10′) according to any one of clauses 22-33, wherein the tube extension section (30, 30′) comprises a plurality of tube extension sub-sections (30a, 30b, 30c, 30d, 30e, 30′a, 30′b, 30′c), and the at least one connector (40a, 40b, 40c, 40d, 40e, 40′a, 40′b, 40′c) is a plurality of connectors (40a, 40b, 40c, 40d, 40e, 40′a, 40′b, 40′c), each connector (40a, 40b, 40c, 40d, 40e, 40′a, 40′b, 40′c) being associated with a corresponding tube extension sub-section (30a, 30b, 30c, 30d, 30e, 30′a, 30′b, 30′c).
35. The medicament tube arrangement (10, 10′) according to clause 34, wherein each connector (40a, 40b, 40c, 40d, 40e, 40′a, 40′b, 40′c) comprises a main body (141) being breakable upon being subject to a pulling force larger than a threshold value, and wherein the threshold value varies for the different main bodies (141) of the connectors (40a, 40b, 40c, 40d, 40e, 40′a, 40′b, 40′c).
36. The medicament tube arrangement (10, 10′) according to any one of clauses 21-34, further comprising a housing (60) arranged to cover the tube extension section (30, 30′).
37. The medicament tube arrangement (10, 10′) according to clause 36, wherein the housing (60) comprises a first opening (60a), and wherein the downstream tube section (20a, 20′a) extends through the first opening (60a) such that a predefined length of the flexible tube (20, 20′) is available outside of the housing (60).
38. An infusion set (1, 10′, 501) comprising a medicament delivery device and a medicament tube arrangement (10, 10′, 510, 610, 710) according to any one of the preceding clauses.

1-15. (canceled)

16. A medicament tube arrangement for an infusion set, comprising:
a first flexible tube structure and a second flexible tube structure for delivering medicament from a medicament reservoir; and
at least one connector being configured to connect the first flexible tube structure to the second flexible tube structure,
wherein the connector is arrangeable in a first configuration in which the first flexible tube structure is arranged adjacent the second flexible tube structure, and is arrangeable in a second configuration in which the first flexible tube structure is arranged spaced apart from the second flexible tube structure.

17. The medicament tube arrangement according to claim 16, wherein the connector is configured to extend the radial reachability of medicament tube arrangement.

18. The medicament tube arrangement according to claim 16, wherein at least a part of the connector is configured to be in contact with the first flexible tube structure and the second flexible tube structure in both the first and second configurations.

19. The medicament tube arrangement according to claim 16, wherein the connector is configured to extend the axial reachability of medicament tube arrangement.

20. The medicament tube arrangement according to claim 16, further comprising:
a first flexible tube forming the first flexible tube structure for delivering medicament from a first medicament reservoir, the first flexible tube having a first tube lumen configured to transport the medicament from the first reservoir;
a first tube locking protrusion forming the connector, the first tube locking protrusion forming a radial protrusion of the first flexible tube; and
a second flexible tube forming the second flexible tube structure for delivering medicament from a second medicament reservoir, the second flexible tube having a second tube lumen configured to transport the medicament from the second reservoir, and a second tube receiving recess configured to releasably lock to the first tube locking protrusion to co-route the first flexible tube with the second flexible tube.

21. The medicament tube arrangement according to claim 20, wherein along a first portion of the medicament tube arrangement, the first tube locking protrusion is locked to the second tube receiving recess such that a first portion of the first flexible tube is co-routed with a corresponding first portion of the second flexible tube.

22. The medicament tube arrangement according to claim 21, wherein along a second portion of the medicament tube arrangement, the first tube locking protrusion is distant from the second tube receiving recess such that a second portion of the first flexible tube is freely arrangeable in relation to a corresponding second portion the second flexible tube.

23. The medicament tube arrangement according to claim 21, further comprising a lock-and-release unit having a main body movable along the first and second flexible tubes in a forward direction and a backward direction, the main body having a first through-hole through which the first flexible tube extends and a second through-hole through which the second flexible tube extends, wherein the first and second through-holes are separated by a spacing, and wherein the spacing is sized such that the first tube locking protrusion of the first flexible tube locks to the second tube receiving recess of the second flexible tube when the main body is moved in the forward direction, and such that the first tube locking protrusion the first flexible tube is released from the second tube receiving recess of the second flexible tube when the main body is moved in the backward direction.

24. A medicament tube arrangement for an infusion set according to claim 1, comprising:
a flexible tube for delivering medicament, the flexible tube comprising the first flexible tube structure and the second flexible tube structure, the flexible tube having a tube length arranged to extend between the medicament reservoir and an injection or infusion site, and a tube extension section dividing the flexible tube into at least an upstream tube section arranged to extend between the medicament reservoir and the tube extension section, and a downstream tube section arranged to extend between the tube extension section and the infusion or injection site,
wherein the connector is arranged in the tube extension section, the connector being configured to, upon being subject to a pulling force larger than a threshold value, re-configure the tube extension section and thereby extend the reachability of the upstream tube section and/or the downstream tube section.

25. The medicament tube arrangement according to claim 24, wherein the connector has a main body comprising a first end portion having a first contact surface, and a second end portion arranged opposite to the first end portion, the second end portion having a second contact surface, wherein the first contact surface is attached to a first connecting portion of the flexible tube in the tube extension section, the first connecting portion forming the first flexible tube structure, and the second contact surface is attached to a second connecting portion of the flexible tube in the tube extension section, the second connecting portion forming the second flexible tube structure, wherein the first and second contacting portions of the flexible tube in the tube extension section are distant from each other.

26. The medicament tube arrangement according to claim 25, wherein the tube extension section is defined to extend from the first connecting portion to the second connecting portion.

27. The medicament tube arrangement according to claim 25, wherein the connector comprises a first tubular tube holder encompassing the first contacting portion of the flexible tube, the first tubular tube holder comprising the first contact surface, and a second tubular tube holder encompassing the second contacting portion of the flexible tube, the second tubular tube holder comprising the second contact surface.

28. The medicament tube arrangement according to claim 25, wherein the first connecting portion and the second connecting portion are separated by a loop of the flexible tube, or a tube bend of the flexible tube.

29. The medicament tube arrangement according to claim 25, wherein the main body of the connector is stretchable.

30. The medicament tube arrangement according to claim 25, wherein the main body of the connector is breakable.
